(19) United States
(12) Patent Application Publication
(10) Pub. No.: US 2025/0218572 A1
(43) Pub. Date: Jul. 3, 2025

(54) NON-TRANSITORY COMPUTER-READABLE MEDIUM STORING PROGRAM

(71) Applicant: CureApp, Inc., Tokyo (JP)

(72) Inventors: Shinya FUTAGAMI, Tokyo (JP); Shotaro TAUCHI, Tokyo (JP); Masahiko KUBARA, Tokyo (JP)

(73) Assignee: CureApp, Inc., Tokyo (JP)

(21) Appl. No.: 18/966,369

(22) Filed: Dec. 3, 2024

(30) Foreign Application Priority Data
Dec. 27, 2023 (JP) 2023-221179
Sep. 9, 2024 (JP) 2024-155032

(51) Int. Cl. G16H 30/20; G16H 10/60

(52) U.S. Cl. CPC G16H 30/20; G16H 10/60

(57) ABSTRACT

A technology to support a record of measured biological information is provided. A non-transitory computer readable medium storing a program that causes a computer of a user terminal to execute; acquiring an analysis result of a picture which images a measurement instrument in every measurement session as a measured value, the measurement instrument measuring biological information, displaying the acquired measured value on a display section of the user terminal, associating with the picture of an acquiring source, and accepting an amendment of the measured value through an operation for the user terminal.

### CROSS REFERENCE TO RELATED APPLICATIONS

This application is based on and claims priority under 35 USC § 119 to Japanese Patent Application No. 2023-221179 filed Dec. 27, 2023 and Japanese Patent Application No. 2024-155032 filed Sep. 9, 2024, the disclosure is incorporated herein by reference in its entirety.

### BACKGROUND

### Technical Field

The present disclosure relates to a non-transitory computer-readable medium storing a program.

### Related Art

In the therapy for high blood pressure guideline 2019, it is recommended that home blood pressure be measured twice each morning and night. Therefore, in a prescription digital therapeutic application for hypertension prescribed for a patient (hereinafter referred to as “therapeutic app”), two input fields in each morning and night are provided.

### CITATION LIST

### Patent Literature

PTL 1: Japanese Patent Application Laid-Open Publication No. 2013-226257

Totally four recording works, two in the morning and two at night, are burdens for a patient who has never had a habit of measuring the home blood pressure, and there is a concern that measurement and recording of the home blood pressure are not continued. As one of the strategies to reduce a workload of a user, there is a health management application using a camera function of a smartphone. When using this type of the health management application, just by imaging a liquid crystal screen of a sphygmomanometer, it is possible to read and record a measured value automatically.

However, in this type of the health management application, kinds of sphygmomanometers that are able to read a picture are limited. Incidentally, reading accuracy depends on imaging quality of the picture. Therefore, when imaging quality is low, a read value becomes a wrong numerical value.

### SUMMARY

An aspect of the present disclosure whose one of purposes is to provide a technology to support a record of measured biological information.

An aspect of the present disclosure provides a non-transitory computer readable medium storing a program that causes a computer of a user terminal to execute; acquiring an analysis result of a picture which images a measurement instrument in every measurement session as a measured value, the measurement instrument measuring biological information, displaying the acquired measured value on a display section of the user terminal, associating with the picture of an acquiring source, and accepting an amendment of the measured value through an operation for the user terminal.

According to the aspect of the present disclosure, it is possible to support a record of measured biological information.

### BRIEF DESCRIPTION OF DRAWINGS

FIG. 1 illustrates a configuration example of a user terminal assumed in Exemplary embodiment 1;

FIG. 2 illustrates a relationship between a measurement timing and a record timing assumed in Exemplary embodiment 1;

FIG. 3 illustrates an image of display screen in a sphygmomanometer by the user terminal;

FIG. 4 illustrates an example of a processing operation performed in the user terminal used in Exemplary embodiment 1;

FIG. 5 illustrates an example of a home screen of the therapeutic app assumed in Exemplary embodiment 1;

FIG. 6 illustrates an example of a confirmation screen of read value which is displayed when an operation button is operated;

FIG. 7 shows a display example of a home screen which is displayed when a storing button is operated in the confirmation screen;

FIG. 8 illustrates an example of an editing screen which is displayed when an “Edit” button is operated in any reading information fields displayed in the confirmation screen;

FIG. 9 illustrates a display example of the confirmation screen describing a state that corresponding reading information field is deleted by operating “Delete” button;

FIG. 10 illustrates a relationship between a measurement timing and a record timing assumed in Exemplary embodiment 2;

FIG. 11 an example of a processing operation performed in the user terminal used in Exemplary embodiment 2;

FIG. 12 illustrates an example of a home screen of the therapeutic app assumed in Exemplary embodiment 2;

FIG. 13 illustrates an example of a measurement screen;

FIG. 14 illustrates an example of a camera output screen and a read value confirming screen;

FIG. 15 shows a display example of a home screen at a time when recording home blood pressure in the morning is finished;

FIG. 16 illustrates a configuration example of information processing system assumed in Exemplary embodiment 3;

FIG. 17 illustrates an example of a processing sequence performed in the user terminal used in Exemplary embodiment 3;

FIG. 18 illustrates an example of a selection screen of an analysis object assumed in Exemplary embodiment 4;

FIG. 19 illustrates an example of a mercury column sphygmomanometer reading a blood pressure value from height of a mercury;

FIG. 20 illustrates a case when the measurement instrument is an analog type body weight;

FIG. 21 illustrates another display example of the selection screen used for selecting a picture of the analysis object;

FIG. 22 illustrates another display example of the selection screen used for selecting a picture of the analysis object;

FIG. 23 illustrates another display example of the selection screen used for selecting a picture of the analysis object;

FIG. 24 illustrates another display example of the camera output screen and the read value confirming screen;

FIG. 25 illustrates a transition screen from the camera output screen to the read value confirming screen.

### DETAILED DESCRIPTION

### Term

Firstly, terms used in Exemplary embodiments to be mentioned later are described.

“Therapeutic app” is an application approved by the Pharmaceutical and Medical Device Act. In the therapeutic app, there are an application executed in terminal operated by a doctor, and an application executed in terminal operated by a patient (in other words, a patient terminal). The patient terminal is an example of the user terminal.

In the Exemplary embodiments described later, the “therapeutic app” is used in meaning of the application executed in terminal operated by a patient. Note that, in the following description, as long as not being unfastidious specifically, the “patient” is written as a user.

The therapeutic app is used for recording of biological information and mood or the like measured at outside of medical institutions. The therapeutic app is available from an application store.

Note that, a disease that obtains an approval of the therapeutic app is, for example, high blood pressure, nicotine addiction and insomnia. In addition, a disease that the therapeutic app is under development is, for example, Non-Alcoholic Steatohepatitis (NASH) and a kidney disease.

“A health management application” is an application that is not approved in the Pharmaceutical and Medical Device Act and an application used for recording of biological information. Therefore, the user of the health management application is not limited to so-called a patient.

The health management application is executed in terminal operated by the user (in other words, a user terminal). The health management application is also available from the application store. The health management application is also used for recording of biological information and mood or the like.

The therapeutic app and the health management application are examples of application program performed in the user terminal for the user (including the patient) to record his/her biological information.

“Biological information” is information measured from a body through a measurement instrument, for example, blood pressure, pulse, breathing, body temperature, body weight, blood sugar level, Na/K, oxygen saturation and brain waves.

“Measurement instrument” is an instrument used in measuring the biological information. The instrument is classified, for example, into a health management instrument or a medical instrument. The measurement instrument includes, for example, an automatic electronic sphygmomanometer, a manual electronic sphygmomanometer, an electronic thermometer, a urine sugar meter for self-testing, a self-measurement instrument of blood sugar, Na/K scan, a pulse oximeter, a body weight meter, a body fat meter, a body composition meter, an activity amount meter, a pedometer and a heartbeat meter.

In case of the exemplary embodiment described below, the “medical institution” is assumed to be an insurance medical institution. More specifically, the medical institution is assumed to be an insurance medical institution to which a doctor who issues prescription code necessary for activating the therapeutic app belongs.

However, by deregulation or the like, when pharmacists, public health nurses, nurses, nutritionists, hospital staff and other medical personnel are able to issue prescription code, medical institution includes facility and institution in which there are above medical personnel.

Note that, issue of prescription code may be not only issue by medical therapy but also issue by out of insurance (i.e. free medical therapy) or issue by mixed medical therapy. Incidentally, the medical therapy includes not only face-to-face medical therapy but also online medical therapy.

### Exemplary Embodiment 1

Hereinafter, exemplary embodiment 1 of the present disclosure will be described with reference to the drawings.

### Device Configuration

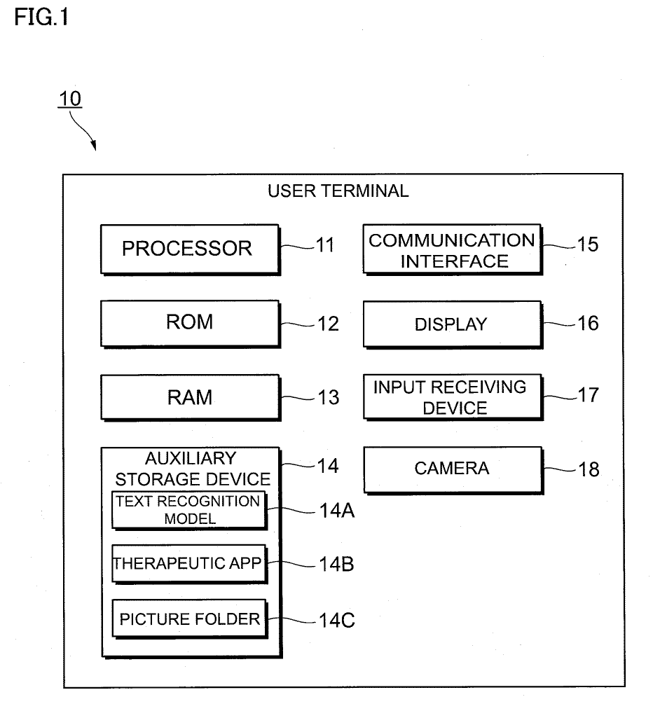

FIG. 1 illustrates a configuration example of a user terminal 10 assumed in the Exemplary embodiment 1.

The user terminal 10 is an information terminal operated by the user and the therapeutic app is installed in the user terminal 10. Therefore, the user terminal 10 in the exemplary embodiments is an information terminal operated by a patient who is prescribed the therapeutic app.

The user terminal 10 in the exemplary embodiments is a smartphone.

The user terminal 10 shown in FIG. 1 includes a processor 11, a Read Only Memory (ROM) 12 storing a Basic Input Output System (BIOS), etc., a Random Access Memory (RAM) 13 used as a work area of the processor 11, an auxiliary storage device 14, a communication interface 15, a display device 16, an input receiving device 17 and a camera 18. Each device is connected via a not-shown signal wire.

The processor 11, ROM 12 and RAM 13 configure so-called a computer.

In the exemplary embodiments, the auxiliary storage device 14 is a rewritable nonvolatile semiconductor memory. The auxiliary storage device 14 may be an internal storage or an external storage.

In case of FIG. 1, in the auxiliary storage device 14, a text recognition model 14A, a therapeutic app 14B and a picture folder 14C are stored.

The text recognition model 14A is a machine learned model that outputs a character string from a picture imaged in the camera 18. The text recognition model 14A includes a preprocessing section that extracts a partial picture including a character string from a picture which is a processing object, and a character conversion section that regards the partial picture extracted in the preprocessing section as an input, and regards corresponding character string as an output.

The text recognition model 14A in the exemplary embodiment is downloaded from an application store when the therapeutic app 14B is downloaded. However, the text recognition model 14A may be a part of the therapeutic app 14B.

In the exemplary embodiment, as the therapeutic app 14B, an application prescribed to a patient of high blood pressure is assumed. Hereinafter, the therapeutic app 14B prescribed to a patient of high blood pressure is referred to as “high blood pressure application”. The high blood pressure application is used for recording of home blood pressure or the like. Note that, home blood pressure or the like recorded in the high blood pressure application is utilized for a therapy in a medical institution.

In the exemplary embodiment, an automatic electronic sphygmomanometer is assumed as a sphygmomanometer used for measuring home blood pressure. In the automatic electronic sphygmomanometer, liquid crystal display for displaying the measured blood pressure value or the like are provided.

In the therapeutic app 14B, by cooperating with the text recognition model 14A, a function is prepared, which performs automatic input of blood pressure value displayed on the liquid crystal display or the like of the sphygmomanometer. Specifically, the therapeutic app 14B provides the text recognition model 14A with a picture that includes liquid crystal display of the sphygmomanometer as a subject and records the blood pressure value or the like notified from the text recognition model 14A corresponding to the measurement session. The details of processing operation performed by the therapeutic app 14B are described later.

The picture folder 14C is a folder in which a picture file imaged in the camera 18 is stored. The picture file includes data of main body of a picture and attribute information such as date and time of imaging. The attribute information is, for example, Exchangeable Image File Format (Exif) data.

The communication interface 15 is a communication module complies with, for example, Ethernet (registered trademark), a wireless local area network (LAN), a mobile communication system (4G, 5G).

The display device 16 is a display, for example, liquid crystal display or organic Electro Luminescence (EL). The display device 16 is an example of a display section.

The input receiving device 17 is, for example, an electrostatic capacitance type touch sensor or power button. A device which the electrostatic capacitance type touch sensor is installed on a surface of the display device 16 is called a touch panel. The camera 18 includes, for example, a Complementary Metal Oxide Semiconductor (CMOS) sensor.

### Relationship Between Measurement Timing and Record Timing

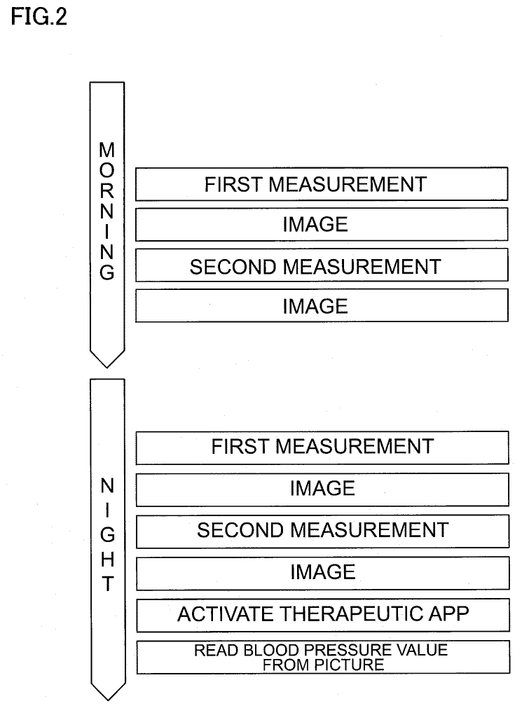

FIG. 2 illustrates a relationship between a measurement timing and a record timing assumed in the Exemplary embodiment 1. As described above, in high blood pressure guideline 2019, it is recommended to measure the home blood pressure totally four times, two in the morning and two at night.

Therefore, also in FIG. 2, the home blood pressure is measured for two times in the morning, and two times at night. In the exemplary embodiment, the user images measured value displayed on a display screen in the sphygmomanometer by the camera 18 (refer to FIG. 1) for every time when measuring blood pressure by the sphygmomanometer.

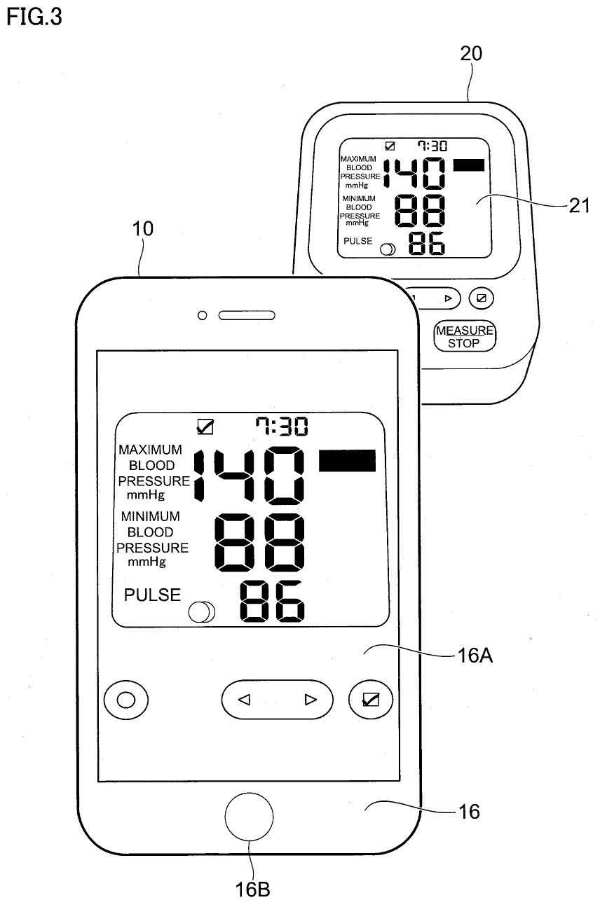

FIG. 3 illustrates an image of a display screen 21 in a sphygmomanometer 20 by the user terminal 10. In the exemplary embodiment, the user activates a camera application when imaging the display screen 21. The camera application is a different application from the therapeutic app 14B (refer to FIG. 1).

On the display device 16 of the user terminal 10 shown in FIG. 3, operation screen of the activated camera application is displayed. Note that, on an imaging area 16A, a picture of the display screen 21, which the camera 18 (refer to FIG. 1) is imaging, is displayed.

When an imaging button 16B is tapped, at the time of the tapping, the picture being displayed on the imaging area 16A is stored in a picture folder 14C (refer to FIG. 1), as a picture file. Note that, the imaging button 16B may be a hard key or a soft key.

In FIG. 3, systolic blood pressure (hereinafter referred to as “maximum blood pressure”) is 140 mmHg, diastolic blood pressure (hereinafter referred to as “minimum blood pressure”) is 88 mmHg and pulse is 86 times/minutes.

Return to the description of FIG. 2.

The picture of the display screen 21 (refer to FIG. 3) is imaged at least one time in every measurement session. Therefore, there may be plural pictures of the display screen 21 in every measurement session.

In the exemplary embodiment, the user activates only the camera application in two times of measuring in the morning. In other words, the user does not activate the therapeutic app 14B (refer to FIG. 1).

Therefore, in time zone of morning, the user simply images and records the picture of the display screen 21 corresponding to every measurement session.

Even in the two times of measuring at night, the user activates the camera application and images a picture of the display screen 21 in every measurement session. Thus far, it is same as the time of measuring in the morning, however, in the measuring at night, the therapeutic app 14B (refer to FIG. 1) is activated after imaging the picture of the display screen 21, and reading of blood pressure value from the picture is instructed. In other words, the user, who operates the therapeutic app 14B described in the exemplary embodiment, records 4 measured values of the home blood pressure together.

As described below, when it is not necessary to amend a numerical value of the blood pressure value read from the picture imaged from the display screen 21, operations that the user is required to perform are, to tap the imaging button 16B (refer to FIG. 3), to operate the therapeutic app 14B to activate, and to instruct to read the blood pressure value.

Incidentally, for two times in the morning and night, when the maximum blood pressure, the minimum blood pressure and the pulse are manually input in every measurement session, activating the therapeutic app 14B, it is necessary to input the numerical value manually in an input field prepared separately according to the measurement session, and the number of operations is more than that of the therapeutic app 14B proposed in the exemplary embodiment.

Note that, when the sphygmomanometer is able to link with Bluetooth, the workload at the time when recording the home blood pressure is smaller than the workload of manual input, however, when malfunction in linking occurs, it is impossible to record the blood pressure value or the like until the malfunction is resolved. Especially, since many of the users who are required to record the home blood pressure are elderly people, there is a possibility that recording the home blood pressure is abandoned due to a reason that a defect is not resolved.

On the other hand, the activation of the therapeutic app 14B provided in the exemplary embodiment may be once a day, and it becomes possible to record the 4 measured values of the home blood pressure together.

### Processing Operation

Hereinafter, on the premise of the relationship shown in FIG. 2, processing operation performed in the user terminal 10 is described.

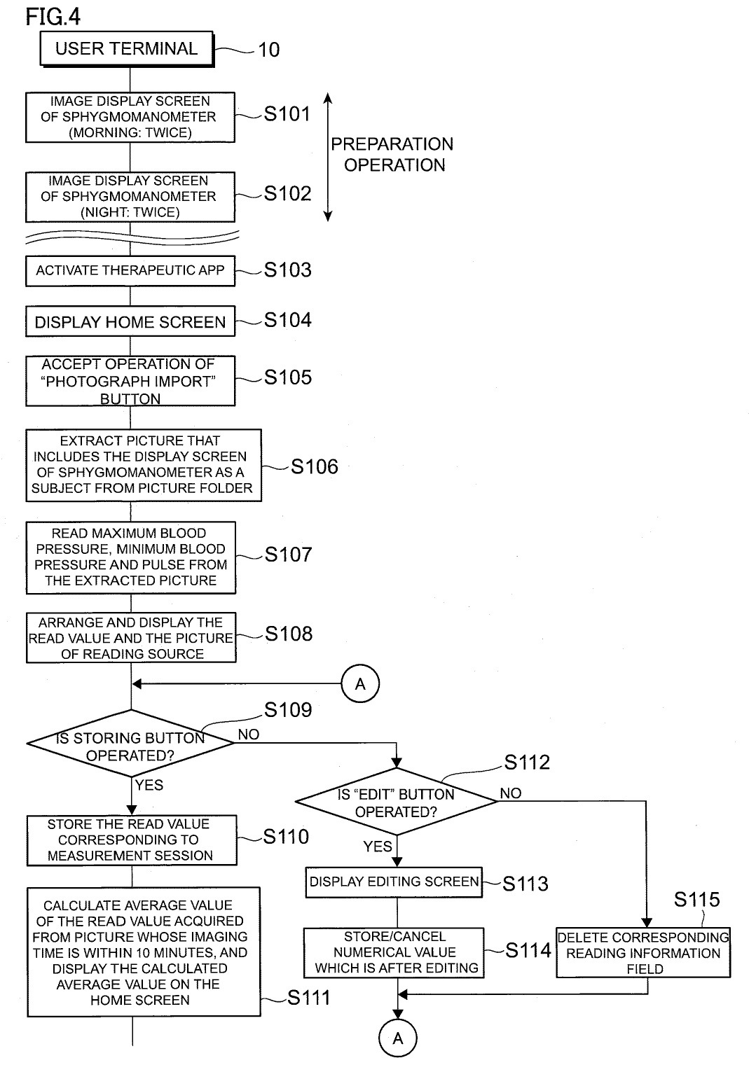

FIG. 4 illustrates an example of a processing operation performed in the user terminal 10 (refer to FIG. 1) used in the Exemplary embodiment 1. The processing operation shown in FIG. 4 is implemented through performances of various kinds of applications by the processor 11 (refer to FIG. 1). The symbol S shown in the figure represents a step.

Firstly, as a preparation operation, the processor 11 images the display screen 21 (refer to FIG. 3) of the sphygmomanometer 20 (refer to FIG. 3), twice in the morning (Step 101). In addition, the processor 11 images the display screen of the sphygmomanometer 20, twice at night (Step 102). Note that, in imaging the display screen 21, the camera application is used. When imaging the measured value for totally four times in the morning and night are finished, the processor 11 activates the therapeutic app 14B, based on an operation of the user (Step 103).

Once the therapeutic app 14B is activated, the processor 11 displays a home screen (Step 104).

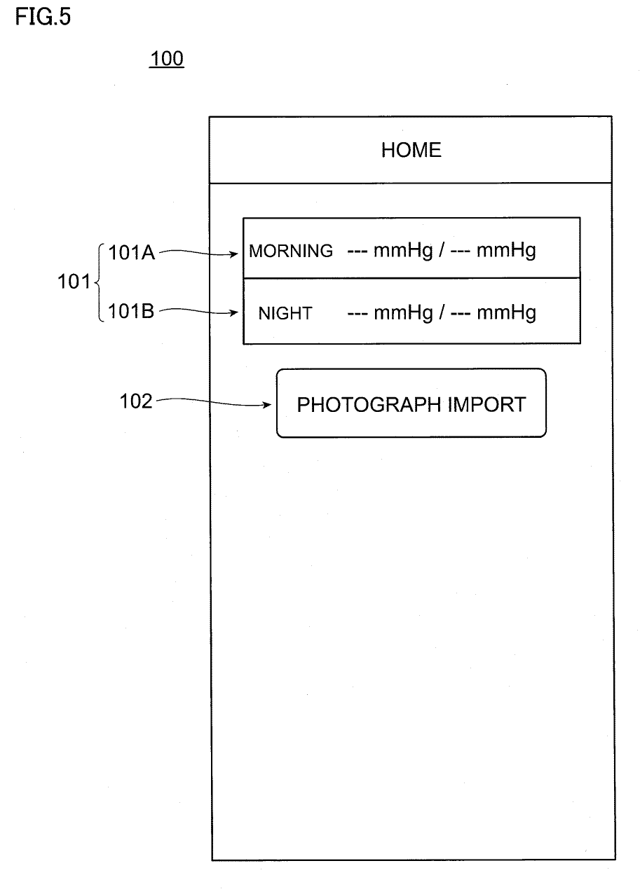

FIG. 5 illustrates an example of a home screen 100 of the therapeutic app 14B assumed in the Exemplary embodiment 1. In the home screen 100 shown in FIG. 5, a display field 101 of an average value of the home blood pressure, and an operation button 102, which instructs to read the blood pressure value from the picture imaging the display screen 21 (refer to FIG. 3), are placed. In case of FIG. 5, in the operation button 102, there is an attached label of “Photograph import”.

In case of FIG. 5, the display field 101 includes a display section 101A of an average blood pressure in the morning, and a display section 101B of an average blood pressure at night.

On the display section 101A of an average blood pressure in the morning, an average value of maximum blood pressure and an average value of the minimum blood pressure, which are read from plural pictures imaged within 10 minutes, are displayed. On the display section 101B of an average blood pressure at night, an average value of the maximum blood pressure and an average value of the minimum blood pressure, which are read from plural pictures imaged within 10 minutes, are displayed.

Note that, when there are plural pictures imaging the display screen 21, whose measuring time is same, the picture read from either one picture may be used in calculation of the average value.

In addition, when there is only one picture imaged in the morning, the maximum blood pressure and the minimum blood pressure, which are read from one picture may be displayed in the display section 101A of an average blood pressure in the morning. The same applies to the display section 101B of an average blood pressure at night.

In case of FIG. 5, since there is not recorded home blood pressure, the display section 101A and the display section 101B are blank.

Return to the description of FIG. 4.

Once the processor 11 accepts an operation of the “Photograph import” button (Step 105), the processor 11 extracts a picture that includes the display screen 21 (refer to FIG. 3) of the sphygmomanometer 20 (refer to FIG. 3) as a subject from the picture folder 14C (refer to FIG. 1) (Step 106).

In the exemplary embodiment, for the purpose of reducing the number of pictures which are objects for reading, in the pictures stored in the picture folder, an object for extraction is only a picture of sphygmomanometer which is not an object for reading yet, and a picture imaged within 24 hours from activation of the therapeutic app 14B.

However, with the result that the recording blood pressure value by activation of the therapeutic app 14B needs to be done only once a day, there is a risk that interest of user for the therapeutic app 14B declines.

Therefore, in the exemplary embodiment, by limiting the object for extraction of the picture to the picture imaged within 24 hours from activation of the therapeutic app 14B, the user is required to activate the therapeutic app 14B for at least once a day. The 24 hours here is an example of a predetermined time.

Note that, it is possible to determine whether the display screen 21 of the sphygmomanometer 20 as a subject is included, by known technology of picture recognition. In the known technology of picture recognition, there is a method to collate between features' patterns prepared in advance and a picture which is an object for determining, based on the display screen 21 of the sphygmomanometer 20. In addition, in the known technology of picture recognition, there is also a method to use a learned model which has performed machine learning on a picture that images the display screen 21 of the sphygmomanometer 20. In the method to use the learned model, the processor 11 provides the learned model with a picture stored in the picture folder 14C (refer to FIG. 1), and acquires an output regarding whether the picture which is a processing object is a picture that includes the display screen 21 of the sphygmomanometer 20 as a subject.

In addition, it is possible to determine whether the picture has been an object for reading, for example, by the presence or absence of a flag added to the picture which is the object for reading.

Moreover, it is possible to determine whether the picture is imaged within 24 hours from activation of the therapeutic app 14B, by reading date and time of imaging recorded in the Exif data that is attribute information of the picture.

Secondly, the processor 11 reads the maximum blood pressure, the minimum blood pressure and the pulse, from the extracted picture (Step 107). Incidentally, the processor 11 provides the text recognition model 14A (refer to FIG. 1) with each extracted picture, and reads out the maximum blood pressure, the minimum blood pressure and the pulse. Furthermore, the processor 11 acquires the date and time of imaging from the Exif data of each picture.

Subsequently, the processor 11 arranges and displays the read value and the picture of a reading source (Step 108).

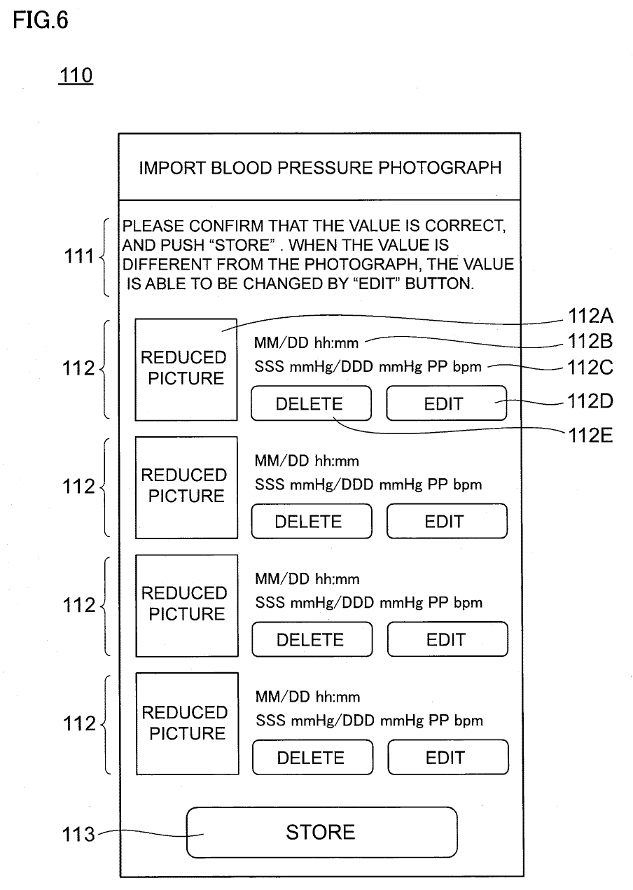

FIG. 6 illustrates an example of a confirmation screen 110 of the read value which is displayed when the operation button 102 (refer to FIG. 5) is operated. In FIG. 6, on the confirmation screen 110 a title of “Import blood pressure photograph” is attached.

On the confirmation screen 110 shown in FIG. 6, a descriptive text 111 describing operation which is required to the user, reading information field 112 corresponding to every measurement session, and a storing button 113 are placed.

In case of FIG. 6, the descriptive text 111 indicates that “Please confirm that the value is correct, and push “Store”. When the value is different from the photograph, the value is able to be changed by “Edit” button ”.

In case of FIG. 6, four reading information fields 112 are displayed. Incidentally, the four reading information fields 112 correspond to each measurement session of two in the morning and two in the night.

However, the number of the reading information fields 112 corresponds to the number of extracted pictures in step 106 (refer to FIG. 4).

Therefore, when the number of pictures extracted in step 106 is, for example two, the number of reading information fields 112 displayed on the confirmation screen 110 is also two. On the other hand, when the number of pictures extracted in step 106 is more than five, the number of reading information fields 112 displayed on the confirmation screen 110 is also five.

However, the number of reading information fields 112 displayed simultaneously on the confirmation screen 110 may be limited to four. In this case, it is possible to change the reading information field 112 displayed on the confirmation screen 110 by a scroll bar.

The reading information field 112 shown in FIG. 6 includes a reduced picture 112A, date and time of imaging 112B, a read value 112C, an “Edit” button 112D, and a “Delete” button 112E, which are of the picture extracted as object for reading.

In the reduced picture 112A, the picture which is a reading source is displayed with reduction. It is desirable that the size of display is able to read the numerical value. However, it may be possible to display the picture with enlargement through a pinch-out, or to display another window whose display size is large, by double-tapping or the like.

In the date and time of imaging 112B, date and time when the picture of a reading source is imaged, is displayed for example, in month (MM)/date (DD) hour (hh): minute (mm). In the read value 112C, maximum blood pressure (SSS mmHg), minimum blood pressure (DDD mmHg) and pulse (PP bpm), which are read from the picture of a reading source, are displayed. In the exemplary embodiment, since the read value 112C is displayed alongside the reduced picture 112A, it is possible for the user to determine whether read numerical value is correct on the same screen.

The “Edit” button 112D is a button to amend the numerical value, when there is an error in the numerical value displayed in the read value 112C. Once the “Edit” button 112D is operated, another window is displayed, and it is possible to amend the maximum blood pressure, the minimum blood pressure and the pulse individually. A layout or the like of another window is described later.

The “Delete” button 112E is a button used to delete the reading information field 112. For example, it is used to delete an unsharp picture, an untargeted picture, and an extra picture which is corresponding to the same measurement session.

In the storing button 113 shown in FIG. 6, there is an attached label of “Store”. Once the storing button 113 is operated, all of the numerical values in the read value 112C displayed on the confirmation screen 110 are finalized.

Return to the description of FIG. 4.

In a state that the confirmation screen 110 (refer to FIG. 6) is displayed, the processor 11 determines whether an operation of the storing button 113 is detected (Step 109).

When the operation of the storing button 113 is detected, a positive result is obtained in step 109. In this case, the processor 11 stores the read value corresponding to measurement session (Step 110).

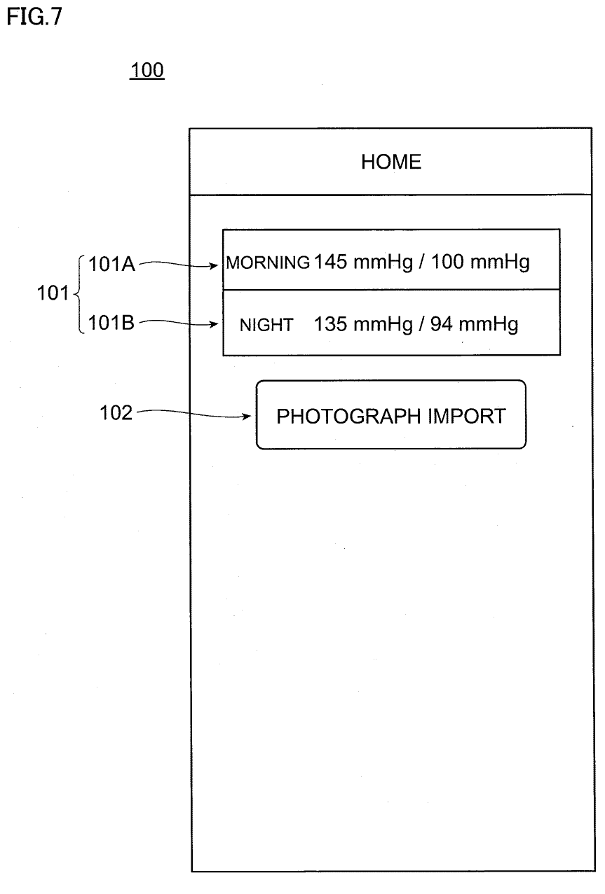

Subsequently, the processor 11 calculates an average value of the read value acquired from a picture whose imaging time is within 10 minutes, and displays the calculated average value on the home screen 100 (refer to FIG. 7) (Step 111). Note that, as described above, when measurement session is only once in one of morning and night, or both, corresponding measured value is displayed on the home screen 100.

FIG. 7 shows a display example of the home screen 100 which is displayed when the storing button is operated in the confirmation screen 110 (refer to FIG. 6). In FIG. 7, components corresponding to those of FIG. 5 are provided with same reference signs.

In case of FIG. 7, the display section 101A of an average blood pressure in the morning indicates that “145 mmHg/100 mmHg”, and the display section 101B of an average blood pressure at night indicates that “135 mmHg/94 mmHg”.

Return to the description of FIG. 4.

Subsequently, a case that a negative result is obtained in step 109 will be described. The case that a negative result is obtained in step 109 is the case that the “Edit” button 112D (refer to FIG. 6) or the “Delete” button 112E (refer to FIG. 6) is operated in any of the reading information fields 112 (refer to FIG. 6).

In case of FIG. 4, the processor 11 determines whether the “Edit” button 112D (refer to FIG. 6) is operated (Step 112). When there is an operation of the “Edit” button 112D, a positive result is obtained in step 112.

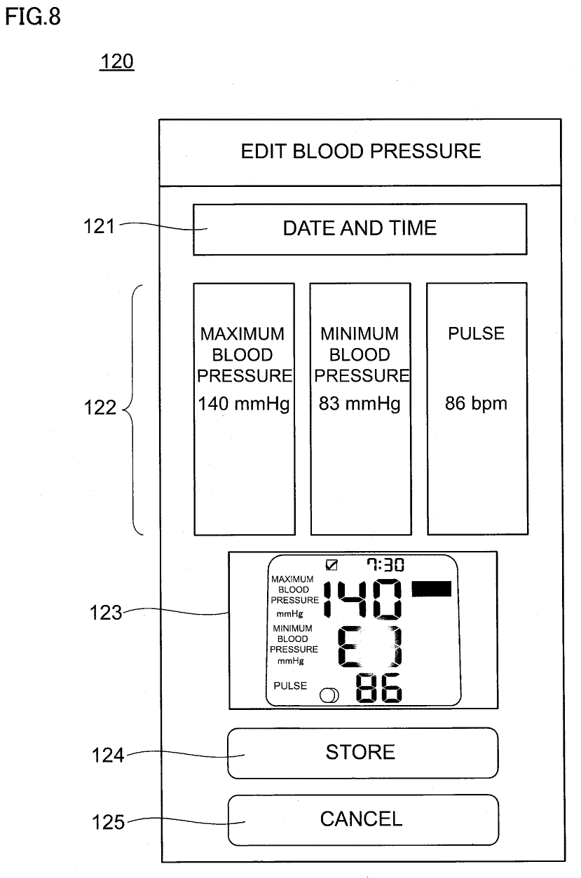

For example, when the user judges that it is necessary to confirm the read numerical value since picture quality of the reduced picture 112A (refer to FIG. 6) is poor, the “Edit” button 112D is operated. In this case, the processor 11 displays an editing screen 120 (refer to FIG. 8) (Step 113). FIG. 8 illustrates an example of an editing screen 120 which is displayed when the “Edit” button 112D is operated in any of the reading information fields 112 displayed in the confirmation screen 110 (refer to FIG. 6).

On the editing screen 120 shown in FIG. 8, a date and time of imaging field 121, an editing accepting field 122, a reading source picture field 123, a storing button 124, and a “Cancel” button 125 are placed.

In the editing accepting field 122, as an initial value, a numerical value of the read value 112C in the confirmation screen 110 (refer to FIG. 6) is displayed. In case of FIG. 8, in editing accepting field 122, “Maximum blood pressure 140 mmHg”, “Minimum blood pressure 83 mmHg” and “Pulse 86 bpm” are displayed.

Incidentally, the display size of the reading source picture field 123 is larger than the reduced picture 112A (refer to FIG. 6). Therefore, the user is able to confirm content of the picture which is reading source more accurately. In case of FIG. 8, in a picture of the display screen 21 (refer to FIG. 3) displayed in the reading source picture field 123, lighting is reflected in a position of a numerical value indicating the minimum blood pressure. Therefore, it is difficult to identify the numerical value, however, it is possible for a person to read that the minimum blood pressure is “88 mmHg”

Note that, when it is possible to display a screen with enlargement by tapping the reading source picture field 123, it is possible to read the numerical value by the picture with more enlargement. In many cases, it is possible to read the numerical value from the editing screen 120 or the screen with enlargement.

In current case, since a read value of the minimum blood pressure is wrong, the user edits the numerical value of the minimum blood pressure in the editing accepting field 122 from “83” to “88” by manual input.

Note that, a picture whose accuracy of text recognition decreases easily, includes an out-of-focus picture, a camera shake picture, other than reflection of environmental light.

Incidentally, when it becomes clear that the read value is correct from the confirmation of the picture displayed on the reading source picture field 123, the user operates the storing button 124 or the “Cancel” button 125, without editing the numerical value of the editing accepting field 122.

Incidentally, in case of FIG. 8, in the storing button 124, there is an attached label of “Store”.

Return to the description of FIG. 4.

When the storing button 124 (refer to FIG. 8) is operated after editing the numerical value of the editing accepting field 122 (refer to FIG. 8), or when the operation of the “Cancel” button 125 (refer to FIG. 8) is accepted, the processor 11 stores the numerical value which is after editing, or cancels the numerical value which is after editing (Step 114). Subsequently, the processor 11 returns to the determination in step 109. In other words, once the processor 11 accepts the operation of the storing button 124 or the “Cancel” button 125, the processor 11 switches a display of the display device 16 (refer to FIG. 1) from the editing screen 120 (refer to FIG. 8) to the confirmation screen 110 (refer to FIG. 6).

Subsequently, a case that a negative result is obtained in step 112 will be described. The case that a negative result is obtained in step 112 is the case that the “Delete” button 112E (refer to FIG. 6) corresponding to any of the reading information fields 112 is operated. In this case, for example, there is a case that a picture is confirmed, which is unrelated to the sphygmomanometer 20 (refer to FIG. 3), from the reduced picture 112A (refer to FIG. 6).

In this case, the processor 11 deletes the corresponding reading information field 112, and returns to step 109 (Step 115).

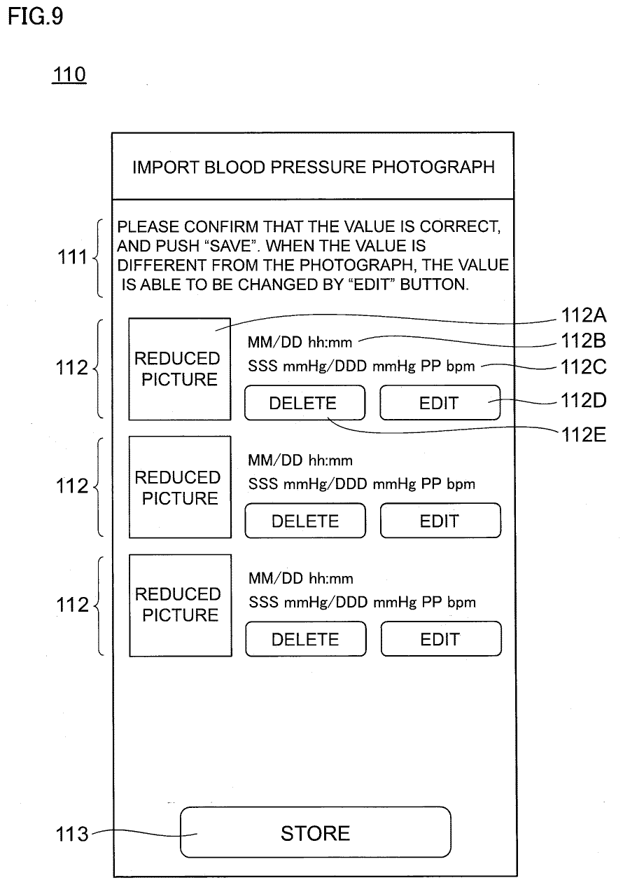

FIG. 9 illustrates a display example of the confirmation screen 110 describing a state that the corresponding reading information field 112 is deleted by operating the “Delete” button 112E. In FIG. 9, components corresponding to those of FIG. 6 are provided with same reference signs.

In the confirmation screen 110 shown in FIG. 6, four reading information fields 112 are displayed, however, the number of reading information fields 112 in the confirmation screen 110 shown in FIG. 9, is three. However, when the number of reading information fields 112 before deleting is more than five, there is a possibility that the number of apparent displaying does not change.

### Brief Conclusion

As described above, when the therapeutic app 14B (refer to FIG. 1) described in the exemplary embodiment is used, it is possible to record together the measurement result of the blood pressure value for totally four times in the morning and night, and it is possible to reduce a workload of the user when recording the home blood pressure.

In addition, since the picture stored in the picture folder is used in the reading source, it becomes possible for a measurement timing and a record timing of the home blood pressure to time shift. As a result, it is possible to realize an effect of reducing a psychological workload of the user recording the home blood pressure. For example, even for the user who feels that it is bothersome to record the home blood pressure in busy time zone before going out, it becomes possible to record the home blood pressure in the therapeutic app without haste after returning home.

Moreover, since imaging a picture which is a reading source of the numerical value is the same as so-called imaging a photograph, it is easy for elderly people to understand it, and it is not necessary to worry about occurrence of malfunction such as linking with Bluetooth. Therefore, it is possible to reduce a risk that the record of the home blood pressure is disconnected due to the occurrence of malfunction in linking with Bluetooth.

Further, in case of the therapeutic app 14B described in the exemplary embodiment, it becomes possible to confirm the picture which is a reading source of the numerical value, and the read value, on the same screen. Therefore, even when quality of the picture which is the reading source of the numerical value is poor, it is possible to discover an error of the read value. Furthermore, in case of the therapeutic app 14B in the exemplary embodiment, when the error of the read value is discovered, since it is possible to amend directly on the screen, it is possible to reduce the number of workloads which is required to the user more than the case of retaking the picture which is the reading source, or remeasuring the home blood pressure.

### Exemplary Embodiment 2

Hereinafter, Exemplary embodiment 2 of the present disclosure will be described with reference to the drawings.

### Device Configuration

Also in the Exemplary embodiment 2, a same user terminal 10 (refer to FIG. 1) as the Exemplary embodiment 1 is assumed. However, a change is made in a part of the function executed in the therapeutic app 14B (refer to FIG. 1).

### Relationship Between Measurement Timing and Record Timing

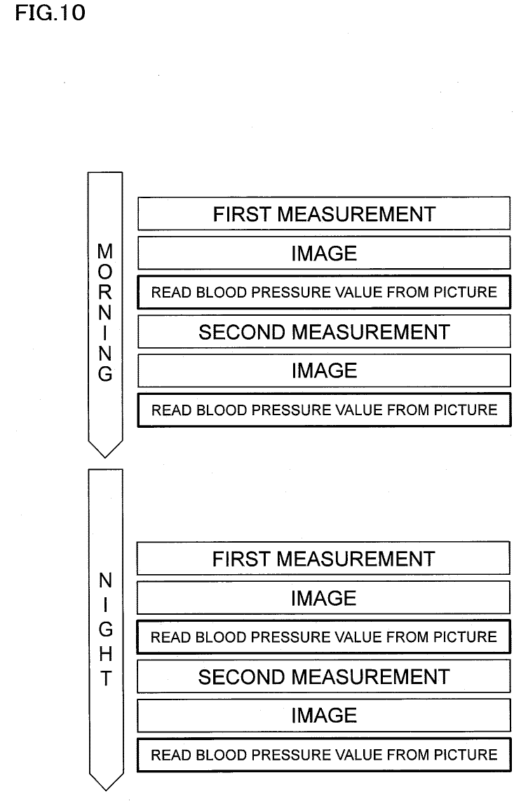

FIG. 10 illustrates a relationship between a measurement timing and a record timing assumed in the Exemplary embodiment 2. The Exemplary embodiment 2 is different from the Exemplary embodiment 1 in terms of which reading of the blood pressure value from the corresponding picture is performed, for every time when imaging the measured value of home blood pressure.

This difference attributes to a difference in a usage form of the therapeutic app 14B (refer to FIG. 1) by the user. In other words, the Exemplary embodiment 2 assumes a user who always activates the therapeutic app 14B, and a user who has little resistance to activating the therapeutic app 14B when measuring the home blood pressure.

Hereinafter, on the premise of the relationship of timing shown in FIG. 10, processing operation performed in the user terminal 10 is described.

### Processing Operation

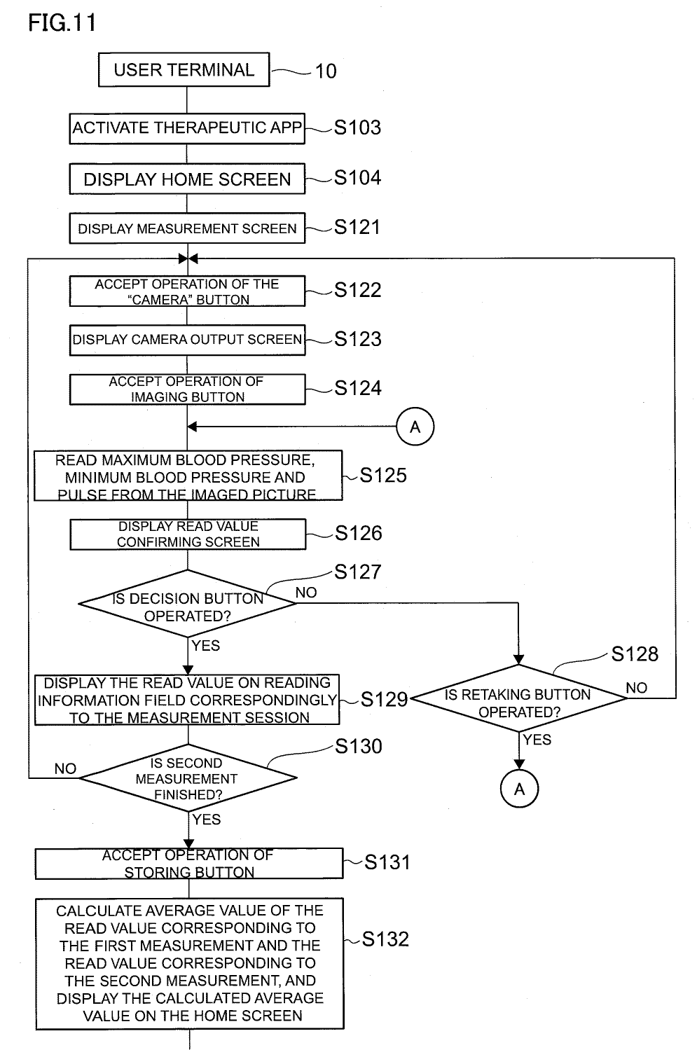

FIG. 11 illustrates an example of a processing operation performed in the user terminal 10 (refer to FIG. 1) used in the Exemplary embodiment 2. In FIG. 11, components corresponding to those of FIG. 4 are provided with same reference signs.

The processing operation shown in FIG. 11 is also implemented through performances of various kinds of applications by the processor 11 (refer to FIG. 1).

The processing operation shown in FIG. 11 is started by an activation (Step 103) of the therapeutic app 14B.

Once the therapeutic app 14B is activated, the processor 11 displays a home screen (Step 104).

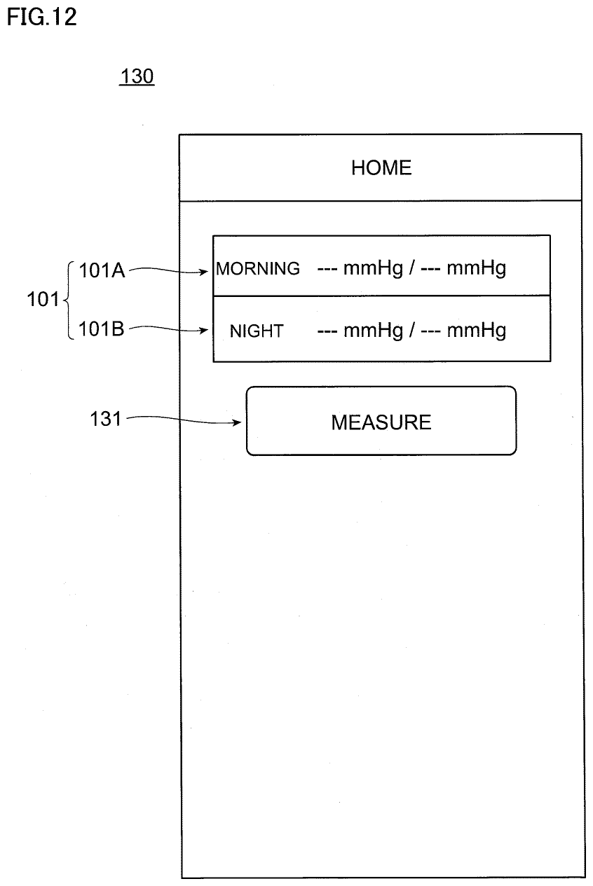

FIG. 12 illustrates an example of a home screen 130 of the therapeutic app 14B assumed in the Exemplary embodiment 2. In FIG. 12, components corresponding to those of FIG. 5 are provided with same reference signs.

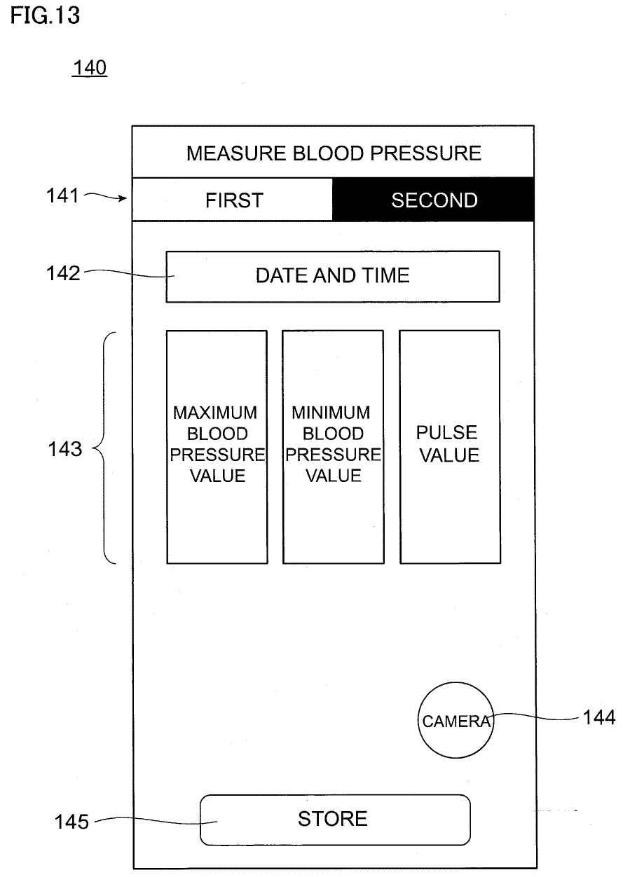

In the home screen 130 shown in FIG. 12, the display field 101 of an average value of the home blood pressure, and an operation button 131 which instructs to display a measurement screen 140 (refer to FIG. 13) are placed.

In case of FIG. 12, in the operation button 131, there is an attached label of “Measure”. In addition, since there is not recorded home blood pressure, the display section 101A and the display section 101B which are shown in FIG. 12, are blank.

Return to the description of FIG. 11.

In the exemplary embodiment, once the processor 11 accepts an operation of the operation button 131 (refer to FIG. 12), the processor 11 displays the measurement screen 140 (refer to FIG. 13) (Step 121).

FIG. 13 illustrates an example of a measurement screen 140. In FIG. 13, in the measurement screen 140, there is an attached label of “Measure blood pressure”.

In the measurement screen 140 shown in FIG. 13, selection field 141 of measurement session, a display field 142 of date and time of measurement, reading information fields 143, a

“Camera” button 144, and a storing button 145 are placed.

In case of the selection field 141 of measurement session in FIG. 13, “First session” is displayed in a highlight state, and “Second” is displayed in a gray-out state. Therefore, the present display is the measurement screen of the first session.

In the reading information fields 143, the home blood pressure or the like read from the picture imaging the display screen 21 (refer to FIG. 3) is displayed. For example, the maximum blood pressure value, the minimum blood pressure value, and the pulse value are displayed. Note that, in case that it is before imaging the display screen 21, numerical values are not displayed in the reading information fields 143.

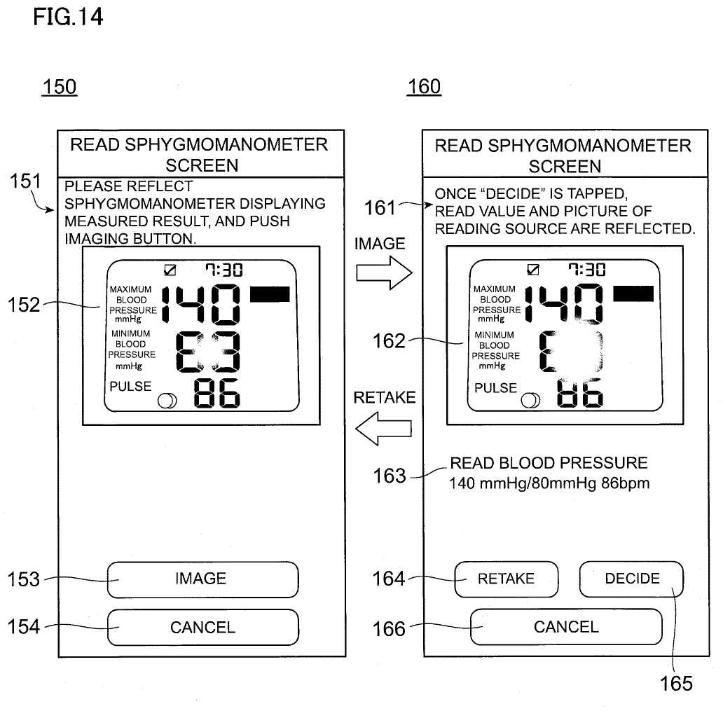

The “Camera” button 144 is a button for instructing to start imaging the display screen 21. Once the “Camera” button 144 is operated, the display of the display device 16 (refer to FIG. 1) is switched to a camera output screen 150 (refer to FIG. 14).

The storing button 145 is a button for recording the blood pressure or the like displayed in the reading information fields 143, correspondingly to the measurement session. In case of FIG. 13, in the storing button 145, there is an attached label of “Store”.

Return to the description of FIG. 11.

Once the processor 11 accepts an operation of the “Camera” button 144 (refer to FIG. 13) (Step 122), the processor 11 displays the camera output screen 150 (refer to FIG. 14) (Step 123).

FIG. 14 illustrates an example of the camera output screen 150 and a read value confirming screen 160.

On the camera output screen 150 shown in FIG. 14, a title of “Read sphygmomanometer screen” is attached.

On the camera output screen 150 shown in FIG. 14, a descriptive text 151 describing operation which is required to the user, a live picture displaying field 152, an imaging button 153, and a “Cancel” button 154 are placed.

In case of FIG. 14, the descriptive text 151 indicates that “Please reflect sphygmomanometer displaying measured result, and push imaging button.”

Note that, the user adjusts a direction or a distance or the like of the camera 18, confirming the display of the live picture displaying field 152. Note that, the picture displayed on the live picture displaying field 152 is temporarily stored in the RAM 13 (refer to FIG. 1) as cache data.

On the imaging button 153 shown in FIG. 14, there is an attached label of “Image”.

Note that, when the “Cancel” button 154 is operated, display of the display device 16 returns to the measurement screen 140 (refer to FIG. 13).

Return to the description of FIG. 11.

Once the processor 11 accepts an operation of the imaging button 153 (refer to FIG. 14) (Step 124), the processor 11 reads the maximum blood pressure, the minimum blood pressure and the pulse (Step 125), from the imaged picture. Incidentally, the processor 11 provides the text recognition model 14A (refer to FIG. 1) with the imaged picture, and reads out the maximum blood pressure, the minimum blood pressure and the pulse.

Subsequently, the processor 11 displays the read value confirming screen 160 (refer to FIG. 14) (Step 126). On the read value confirming screen 160, the read value and the picture of the reading source are displayed alongside

On the read value confirming screen 160 shown in FIG. 14, a descriptive text 161 describing operation which is required to the user, the reading source picture field 162, a read value 163, a retaking button 164, a decision button 165, and a “Cancel” button 166 are placed.

In case of FIG. 14, the descriptive text 161 indicates that “Once “Decide” is tapped, read value and picture of reading source are reflected”. This description means that the user only has to do is to tap the decision button 165 when the read value is correct. In case of FIG. 14, on the decision button 165, there is an attached label of “Decide”.

In the reading source picture field 162 shown in FIG. 14, a picture imaged at a time when the imaging button 153 is tapped is displayed. In case of FIG. 14, in the reading source picture field 162, an effect of lighting is strongly apparent in display position of the minimum blood pressure. The picture displayed in the reading source picture field 162 is cache data. Therefore, the picture displayed in the reading source picture field 162 is read from RAM 13.

In case of FIG. 14, the read value 163 indicates that the minimum blood pressure is “80 mmHg”. However, a positive value of the minimum blood pressure confirmed from the reading source picture field 162 is “88 mmHg”.

The retaking button 164 shown in FIG. 14 is, literally, a button used for instructing to retake the picture which is the reading source. Once the retaking button 164 is operated, the display of the display device 16 is switched to the camera output screen 150.

In case of FIG. 14, on the retaking button 164, there is an attached label of “Retake”. This retaking button 164 is an example of a button used for instructing to amend the measured value.

The decision button 165 is a button to store the read value temporarily, corresponding to the measurement session. Therefore, in a phase that the decision button 165 is operated, the read value is not finalized as the blood pressure value of the measurement session. In other words, the decision button 165 is a button to switch (or renew) the display of the measurement screen 140 (refer to FIG. 13). Note that, in the reading information fields 143 (refer to FIG. 13), the value displayed in the read value 163 at a time when the decision button 165 is operated is displayed.

The “Cancel” button 166 is a button for returning to the measurement screen 140 without storing the read value.

Return to the description of FIG. 11.

The processor 11 determines whether there is an operation of the decision button 165 (refer to FIG. 14) (Step 127), in a state that a read value confirmation screen 160 (refer to FIG. 14) is displayed.

When there is an operation of the retaking button 164 (refer to FIG. 14) or an operation of the “Cancel” button 166 (refer to FIG. 14), a negative result is obtained in step 127. In this case, the processor 11 further determines whether there is the operation of the retaking button 164 (Step 128).

When there is the operation of the retaking button 164, a positive result is obtained in step 128. In this case, the processor 11 returns to step 125. In other words, the processor 11 returns to a display of the camera output screen 150.

On the other hand, there is the operation of the “Cancel” button 166, a negative result is obtained in step 128. In this case, the processor 11 returns to step 122. In other words, the processor 11 returns to a display of the measurement screen 140 (refer to FIG. 13).

Incidentally, when a positive result is obtained in step 127 (in other words, there is an operation of the decision button 165), the processor 11 displays the read value on the reading information field 143 (refer to FIG. 13), correspondingly to the measurement session (Step 129).

Subsequently, the processor 11 determines whether the second measurement finishes (Step 130). When current read value corresponds to the first measurement session, a negative result is obtained in step 130. In this case, the processor 11 returns to step 122. On the other hand, when the current read value corresponds to the second measurement session, a positive result is obtained in step 130. In this case, processor 11 accepts an operation of the storing button 145 (refer to FIG. 13) (Step 131).

Afterward, the processor 11 calculates an average value of the read value corresponding to the first measurement and the read value corresponding to the second measurement, and displays the calculated average value on the home screen 130 (refer to FIG. 12) (Step 132).

Note that, it is possible to calculate the average value only in the case that time difference is within ten minutes, which is between the time when the read value corresponding to the first measurement is stored and the time when the read value corresponding to the second 25 measurement is stored.

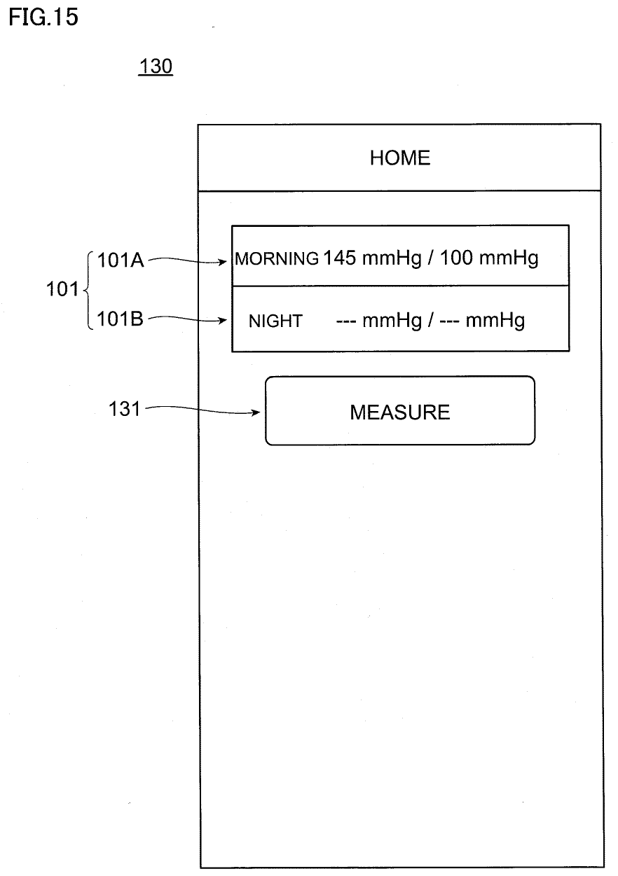

FIG. 15 shows a display example of the home screen 130 at a time when recording home blood pressure in the morning is finished. In FIG. 15, components corresponding to those of FIG. 12 are provided with same reference signs.

In case of FIG. 15, the display section 101A of an average blood pressure in the morning indicates that “145 mmHg/100 mmHg”, and the display section 101B of an average blood pressure at night is still blank.

### Brief Conclusion

As described above, when the therapeutic app 14B (refer to FIG. 1) described in the exemplary embodiment is used, it is possible to read and record a measured value by just imaging the display screen 21 of the sphygmomanometer 20 in every measurement session.

Note that, since it is possible to contrast the picture used in the reading source and the read value on the same screen in a phase prior to recording the blood pressure value read from the picture correspondingly to the measured session, it is also possible to discover an error of the read value easily.

In addition, in the exemplary embodiment, since it is possible to confirm right or wrong in the read value, while the home blood pressure in every measurement session is displayed, when the read value is wrong, it becomes possible to retake the display screen 21 immediately, and record the correct blood pressure value.

### Exemplary Embodiment 3

Hereinafter, Exemplary embodiment 3 of the present disclosure will be described with reference to the drawings.

### System Configuration

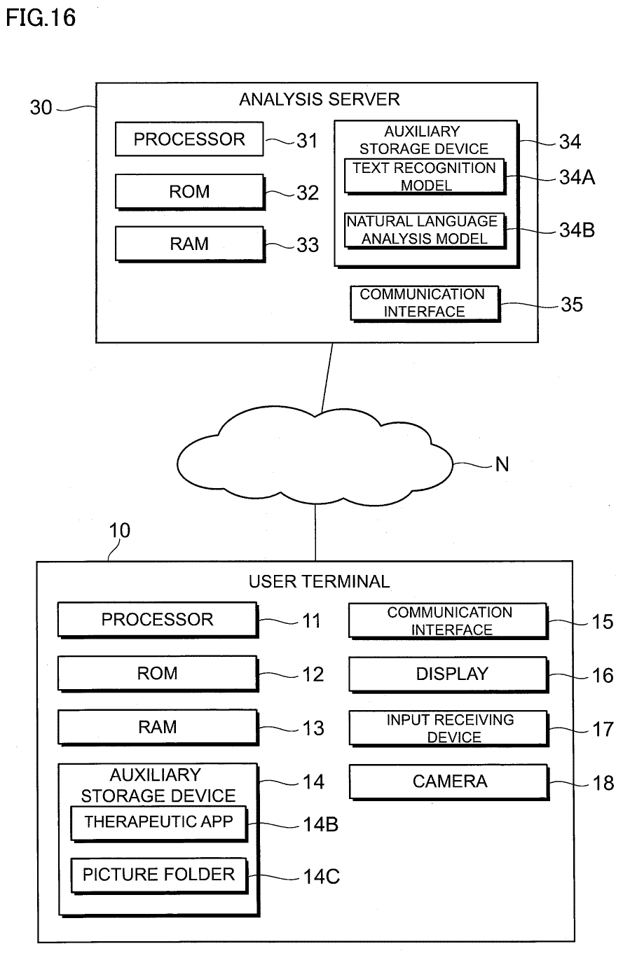

FIG. 16 illustrates a configuration example of information processing system assumed in the Exemplary embodiment 3. In FIG. 16, components corresponding to those of FIG. 1 are provided with same reference signs.

The information processing system shown in FIG. 16 includes the user terminal 10 and an analysis server 30. The Exemplary embodiment 3 is different from the Exemplary embodiment 1 in terms of which the process of reading the blood pressure value from the picture imaging the display screen 21 (refer to FIG. 3) is performed in the analysis server 30, and returns the read value that is an analysis result to the user terminal 10.

Note that, the user terminal 10 and the analysis server 30 are connected to be able to communicate through a network N. The network N is, for example, a local area network (LAN), the Internet, or a mobile communication system (4G, 5G).

In FIG. 16, only one user terminal 10 is connected to the network N, however, plural user terminals 10 may be connected.

In addition, also in the analysis server 30, plural analysis servers 30 may cooperate, execute an analysis of a picture, and notify the user terminal 10 of reading value as an analysis result.

Therefore, in the user terminal 10 used in the exemplary embodiment, the text recognition model 14A is not stored.

The analysis server 30 includes a processor 31, a ROM 32 storing a BIOS, etc., a RAM 33 used as a work area of the processor 31, an auxiliary storage device 34, a communication interface 35. Each device is connected via a not-shown signal wire.

In the auxiliary storage device 34 shown in FIG. 16, a text recognition model 34A or a natural language analysis model 34B is stored.

In the text recognition model 34A used in the exemplary embodiment, it is possible to adopt a model whose accuracy of analysis is higher than the text recognition model 14A (refer to FIG. 1) described in the Exemplary embodiment 1. This is because computational complexity of the analysis server 30 per unit time is significantly larger than the computation volume per unit time of the user terminal 10.

Therefore, in the text recognition model 34A, it is possible to use a model which requires much amount of calculation, but whose accuracy of analysis is high.

The natural language analysis model 34B is a model which has learned not only character pattern but also relationship between characters, and a model whose accuracy of analysis is higher than the text recognition model 34A. However, the natural language analysis model 34B requires even more amount of calculation than the text recognition model 34A. In the auxiliary storage device 34 in FIG. 16, the text recognition model 34A and the natural language analysis model 34B are illustrated, however, in fact, either one is stored.

### Processing Sequence

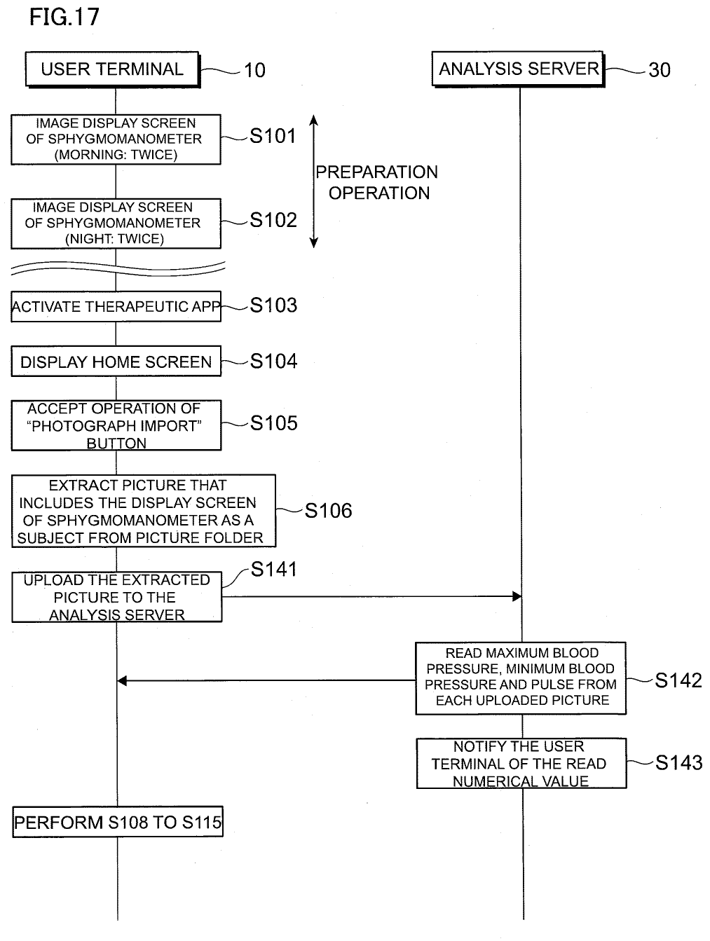

FIG. 17 illustrates an example of a processing sequence performed in the user terminal 10 used in the Exemplary embodiment 3. In FIG. 17, components corresponding to those of FIG. 4 are provided with same reference signs. The processing sequence shown in FIG. 17 is to share an analysis process in the processing operation described in the Exemplary embodiment 1 by the analysis server 30.

Therefore, the processing operations from step 101 to 106 are same as the Exemplary embodiment 1. In other words, once the user images the display screen 21 after finishing the second measurement of the home blood pressure at night, the user activates the therapeutic app 14B (refer to FIG. 1) and instructs to read the home blood pressure.

The processor 11 (refer to FIG. 1) which detects above operation, extracts a picture including the display screen 21 of the sphygmomanometer 20 as a subject from the picture folder (Step 106), and uploads the extracted picture to the analysis server 30 (Step 141). In the exemplary embodiment, at least the pictures of the display screen 21 for totally four times in the morning and night, are uploaded.

The analysis server 30 that is the destination of uploading, reads the maximum blood pressure, the minimum blood pressure and the pulse from each uploaded picture (Step 142).

As described above, the analysis server 30 provides the text recognition model 34A (refer to FIG. 16) or the natural language analysis model 34B (refer to FIG. 16) with the uploaded picture, and reads the blood pressure value or the like from inside of the picture.

Once the reading the blood pressure value or the like is completed, the analysis server 30 notifies the user terminal 10 of the read numerical value (Step 143).

Thereafter, the user terminal 10 performs from step 108 to 115.

### Brief Conclusion

As described above, the therapeutic app 14B (refer to FIG. 1) described in the exemplary embodiment, uploads the picture which is as an analysis object, to the analysis server 30, and acquires the blood pressure value or the like as the analysis result. Since the analysis server 30 is able to use more calculation resources than the user terminal 10, the accuracy of reading the blood pressure value or the like is able to become higher than the case of analyzing the picture at the inside of the user terminal 10. As a result, it is possible to reduce the number of amending numerical values by the user.

Another advantage obtained by analyzing the picture by the analysis server 30 is that type of the display screen 21 which is possible to be read increases.

For example, it is diverse for not only layout of the maximum blood pressure, the minimum blood pressure and the pulse in the display screen 21 of the sphygmomanometer 20 but also type or size of font. In addition, the date of measurement or time is sometimes displayed on the display screen 21.

However, as the exemplary embodiment, through analyzing the picture by the analysis server 30, it becomes possible to extract the blood pressure value or the like from inside of the display screen 21 more accurately.

Note that, in the exemplary embodiment, a processing sequence is described with the processing operation in the Exemplary embodiment 1 as a premise, however, the processing operation in the Exemplary embodiment 2 may be the premise.

### Exemplary Embodiment 4>

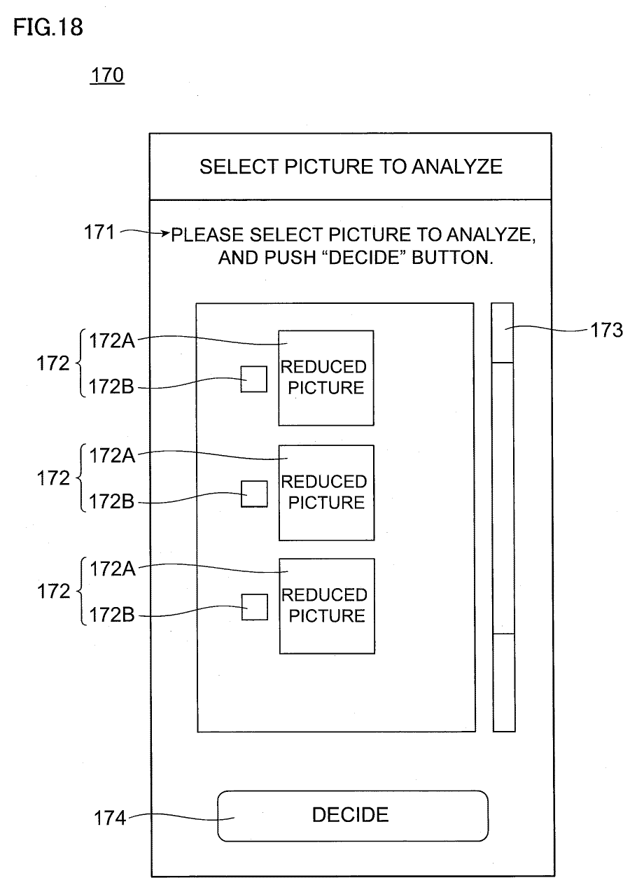

In the Exemplary embodiment 1 described above, all pictures extracted by the therapeutic app 14B based on predetermined condition are set as analysis objects, however, it may be possible for the user to select the picture which is set as an analysis object, in advance. FIG. 18 illustrates an example of a selection screen 170 of an analysis object assumed in Exemplary embodiment 4.

The selection screen 170 shown in FIG. 18, is displayed before the confirmation screen 110 (refer to FIG. 6) is displayed, when the operation button 102 is operated on the home screen 100 (refer to FIG. 5) (in other words, when the “Photograph import” button is operated). On the selection screen 170 shown in FIG. 18, a title of “Select picture to analyze” is attached.

On the selection screen 170 shown in FIG. 18, a descriptive text 171 describing operation which is required to the user, the candidate selecting fields 172 corresponding to the extracted pictures, a scroll bar 173, and a “Decide” button 174 are placed.

In case of FIG. 18, the descriptive text 171 indicates that “Please select picture to analyze, and push “Decide” button.”

The candidate selecting field 172 shown in FIG. 18, includes a reduced picture 172A of an extracted picture, and corresponding check box 172B.

Once the scroll bar 173 is operated, a content of the reduced picture 172A displayed inside the screen, is changed.

As described in the descriptive text 171, the “Decide” button 174 is used to decide the analysis object. After an operation of the “Decide” button 174, the processor 11 provides the text recognition model 14A (refer to FIG. 1) with picture marked check in the check box 172B, and read out the blood pressure value or the like included in the picture. Note that, the processor 11 displays the confirmation screen 110 (refer to FIG. 6) on the display device 16 (refer to FIG. 1), after the analysis is completed.

### Brief Conclusion

In the exemplary embodiment, since the user is able to specify a picture to be set as an analysis object, it is possible to minimize calculation resource used for analysis. In addition, even when additional cost accrues for use of the analysis server 30 (refer to FIG. 16) described in the exemplary embodiment 3, it is possible to minimize the cost required for the analysis by narrowing the number of pictures that desired to be analyzed.

### Other Exemplary Embodiments

(1) So far, the exemplary embodiments of the present disclosure have been described, but the technical scope of the present disclosure is not limited to the scope of the above-described exemplary embodiments. Various modifications or improvements added to the above-described exemplary embodiments may be apparently included in the technical scope of the present disclosure from the description of the claims.

(2) The processor in the above-described exemplary embodiments refers to a processor in a broad sense including, in addition to a general-purpose processor (for example, a Central Processing Unit (CPU)), a dedicated processor (for example, a Graphical Processing Unit (GPU), an Application specific Integrated Circuit (ASIC), a Field Programmable Gate Array (FPGA), a program logic device, etc.).

Moreover, the operation of the processor in each of the above-described exemplary embodiments is not limited to a single processor, but may be performed in cooperation by multiple processors. In addition, the order of execution of each operation in the processor is not limited to the order described above, but may be changed individually.

(3) In the above-described exemplary embodiments, as the user terminal 10 (refer to FIG. 1), a smartphone is illustrated, however, the user terminal 10 may be another terminal when the terminal in which the therapeutic app or the health management application is performed. Another terminal may be, for example, desktop computer, notebook type computer, tablet type computer, or wearable computer. The wearable computer includes, for example, smart glass, head set or smart watch.

(4) In the above-described Exemplary embodiment 1, there is 24-hour limit in the picture which is an object for reading of the blood pressure value, however, the period of limit may be extended, or the limit itself may be eliminated. For example, a picture imaged within 48 hours may be possible to be an object for reading the blood pressure value, or a picture imaged within 1 week may be possible to be an object for reading the blood pressure value. In case of the therapeutic app 14B in the Exemplary embodiment 1, since reading picture only at night or in the morning is enough, there is a possibility that the measurement session which has already elapsed 24 hours appears when the user realizes.

For example, at night, in case that reading the measured value of morning and night in the same day from the picture, even when it is attempted to read the blood pressure value of the previous day in the morning of the next day, it is impossible to import automatically the blood pressure value measured in the morning of the previous day, in the event that the time when the therapeutic app 14B is activated has elapsed 24 hours from the time when the blood pressure value of the morning of the previous day is imaged.

Therefore, the limitation for imaging time of the picture which is an object for reading the blood pressure value, may be more than 24 hours. However, when the picture of the display screen 21, which is imaged previous morning is left, it is possible to input manually the blood pressure value by referring to the picture.

(5) In the above-described Exemplary embodiment 1, a picture imaged within 24 hours from the activation of the therapeutic app 14B is extracted as an object for reading the blood pressure value, however, a picture whose elapsed time from imaging time to present time is within predetermined time (for example, 24 hours) may be an object for reading. Also in this example, the predetermined time may be more than 24 hours (for example, 48 hours, 1 week). In other words, a picture imaged within a predetermined time from operation time of the “Photograph import” button (present time) may be an object for reading.

(6) In the above-described Exemplary embodiment 1, in step 106 (refer to FIG. 4), a picture of an unread sphygmomanometer is extracted, however, only the unread picture may be an object of extracting, only the picture of the sphygmomanometer may be an object of extracting, and these limitations may not be adopted.

(7) In the above-described Exemplary embodiment 1 and 3, a case is described which is a case that home blood pressure is measured in the morning and night for four times in total, and the operation button 102 (refer to FIG. 5) is operated after all pictures corresponding to each measurement session are gathered, however, accepting of the operation for the operation button 102 is unrelated with presence or absence, and number of the pictures corresponding to each measurement session. Therefore, as in the Exemplary embodiment 2, the blood pressure value may be read and recorded from the picture corresponding to each measurement session by operating the operation button 102 at each time when one measurement ends.

(8) In the above-described Exemplary embodiment 2, the processing operation is described, which instructs to retake the display screen 21 (refer to FIG. 3), until correct blood pressure is read, when there is an error in the read value, however, as the Exemplary embodiment 1, it may be possible to amend the read value referring to the displayed picture.

(9) In the above-described exemplary embodiments, it is assumed that the sphygmomanometer 20 includes the display screen 21, and blood pressure value or the like that is a measurement result is displayed on the display screen 21 in digital, however, the measurement result may be displayed in analog.

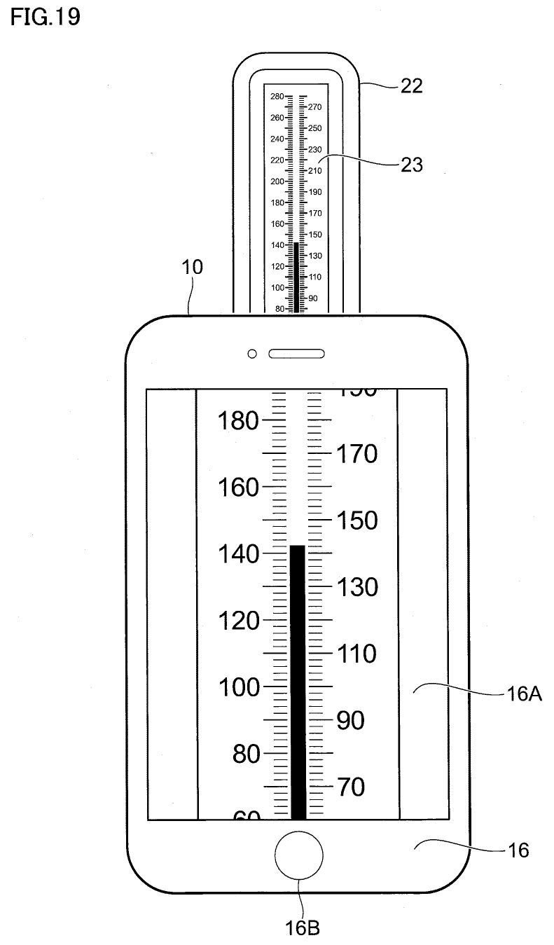

FIG. 19 illustrates an example of a mercury column sphygmomanometer 22 reading a blood pressure value from height of a mercury. In FIG. 19, components corresponding to those of FIG. 3 are provided with same reference signs.

In the mercury column sphygmomanometer 22, height of a mercury column is blood pressure of the user. When the mercury column sphygmomanometer 22 is imaged in the user terminal 10, the processor 11 (refer to FIG. 1) identifies height of tip part of mercury column from a scale 23 engraved along a glass tube, and reads the identified height as the blood pressure value.

Note that, the sphygmomanometer in which the blood pressure value is displayed in analog may be aneroid type that a needle moves along a circular. In a picture of the aneroid type sphygmomanometer, the processor 11 regards a numerical value of needle tip position read from the scale as the blood pressure value.

(10) In the above-described exemplary embodiments, the sphygmomanometer 20 is assumed to be an example of the measurement instrument that measures biological information, however, a technology in the above-described exemplary embodiments is able to be adopted in reading out the measured value from a picture of the measurement instrument other than the sphygmomanometer.

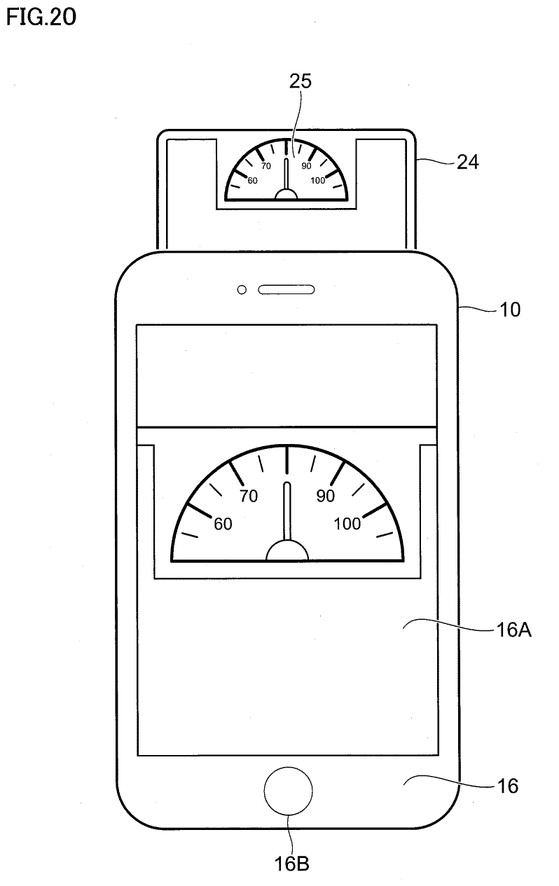

FIG. 20 illustrates a case when the measurement instrument is an analog type body weight 24. In FIG. 20, components corresponding to those of FIG. 3 are provided with same reference signs. Weight of the user is read from a picture imaging a dial face 25 of the analog type body weight. Specifically, the weight of the user is read by the position of the needle for the circularly printed scale.

Note that, in FIG. 20, an analog type body weight meter is illustrated as an example of the measurement instrument, however, the measurement instrument may be a digital type body weight meter.

Moreover, the measurement instrument may be, for example, a clinical thermometer, a urine sugar meter for self-testing, a self-measurement instrument of blood sugar, Na/K scan, a pulse oximeter, a body fat meter, a body composition meter, an activity amount meter, a pedometer, and a heartbeat meter. All of the above may be not limited to a digital type, but also an analog type.

(11) In the above-described exemplary embodiments, a case that a therapeutic app approved by the Pharmaceutical and Medical Device Act is provided with a function to read the measured value from the picture of the measurement instrument is described, however, same function may be provided in the health management application.

(12) In the above-described exemplary embodiments, a case that a picture imaged within predetermined time (for example, within 24 hours from an activation of the therapeutic app) is displayed on the selection screen 170 (refer to FIG. 18) is described.

In this case, the candidate selecting field 172 (refer to FIG. 18) may be sorted separately by date of imaging or time zone of the date of imaging (for example, “morning”, “night”), and displayed. By adopting this displaying function, the user is able to record the blood pressure value effectively for every specific time zone.

(13) In the above-described exemplary embodiments, an upper limit is not set in the selectable number of pictures which is set as the analysis objects of the picture from the candidate selecting field 172 (refer to FIG. 18) which a picture imaged within predetermined time (for example, within 24 hours from an activation of the therapeutic app) is displayed on the selection screen 170 (refer to FIG. 18).

However, in an analysis of a picture, processing load provided to the processor 11 (refer to FIG. 1) is large, and the analysis of a picture also becomes a cause to make waiting time until the analysis result is displayed, be long. Therefore, it may be possible to set an upper limit on the number of pictures which are selectable at one time. For example, four pictures may be the upper limit.

(14) In the above-described exemplary embodiments, a case that a picture imaged within predetermined time (for example, within 24 hours from an activation of the therapeutic app) is displayed on the selection screen 170 (refer to FIG. 18) is described. In case of the above-described exemplary embodiments, since it is possible to operate the “Photograph import” button repeatedly, even a picture which is selected as an object for reading the measured value in a previous operation, is displayed as a selection candidate.

However, when a picture from which blood pressure value or the like has been failed to be read is displayed as a reading candidate every time, the user is lead to perform a useless selecting work repeatedly. In addition, it also results in wasting calculation resource such as the processor 11 (refer to FIG. 1) to repeat the analysis of the picture failed to be read. Moreover, there is no need to read repeatedly a picture which has succeeded in reading blood pressure value or the like previously.

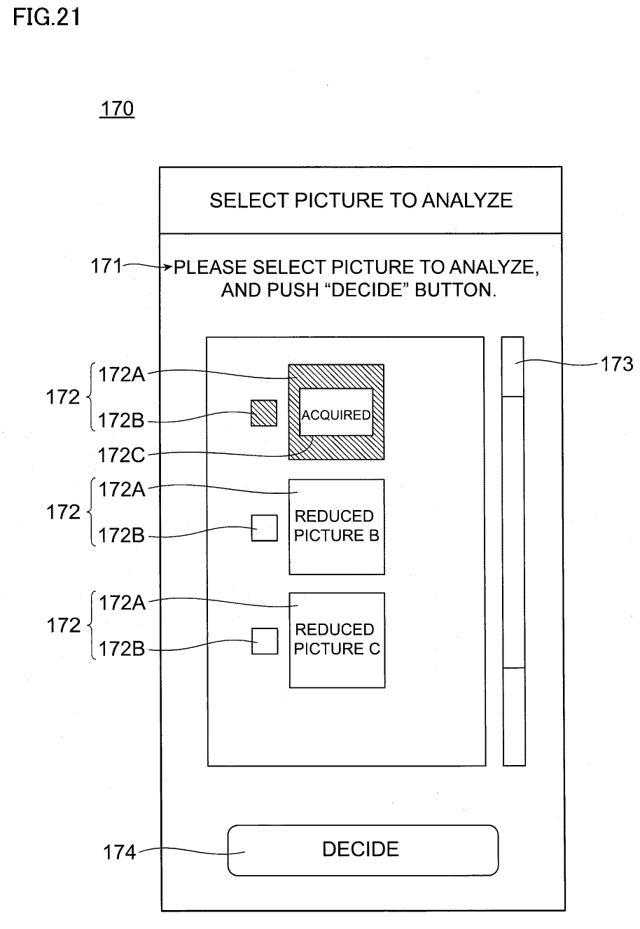

FIG. 21 illustrates another display example of the selection screen 170 used for selecting a picture of the analysis object. In FIG. 21, components corresponding to those of FIG. 18 are provided with same reference signs.

In case of FIG. 21, three reduced pictures 172A are written as a “reduced picture A”, a “reduced picture B” and a “reduced picture C” in an order from top to bottom in FIG. 21. However, the reduced picture 172A corresponding to the “reduced picture A” is displayed in a gray-out state. In addition, a label 172C showing “acquired” is displayed in a pop-up state on foreground side of the reduced picture 172A. Incidentally, the “acquired” is an example, and it is possible to display “analyzed”, “read”, “unselectable” and the like. Further, check box 172B corresponding to the “reduced picture A” is also displayed in a gray-out state. Note that, the “reduced picture A” here is an example of a first candidate.

Due to these displays, it is possible for the user to realize that the “reduced picture A” has already been selected as the analysis object, while the “reduced picture A” is a picture imaged within predetermined time.

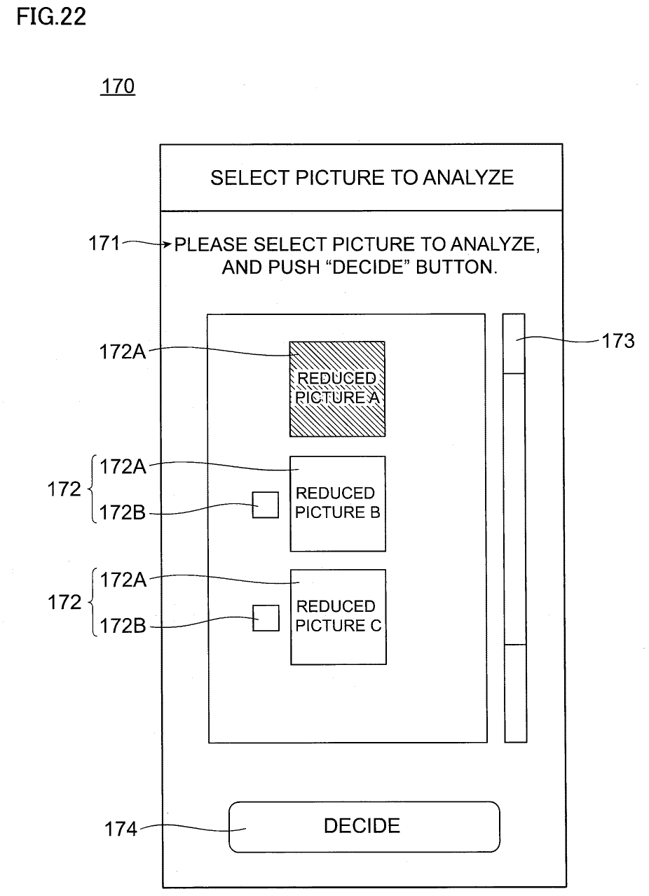

FIG. 22 illustrates another display example of the selection screen 170 used for selecting a picture of the analysis object. In FIG. 22, components corresponding to those of FIG. 18 are provided with same reference signs.

Also in case of FIG. 22, the three reduced pictures 172A are written as a “reduced picture A”, a “reduced picture B” and a “reduced picture C” in an order from top to bottom in FIG. 22.

In case of FIG. 22, the reduced pictures 172A corresponding to the “reduced picture A” is displayed in a gray-out state, and the corresponding check box 172B is not displayed.

Since the reduced pictures 172A is displayed in a gray-out state, it is possible for the user to realize that the “reduced picture A” has already been selected as the analysis object, while the “reduced picture A” is a picture imaged within predetermined time.

Note that, since the label 172C (refer to FIG. 21) is not displayed, the user is able to confirm a content of the “reduced picture A”, while in the condition that visibility is weak due to displaying in a gray-out state. Therefore, the user is helpful for selecting the “reduced picture B” and the “reduced picture C” or the like.

In addition, in case of the selection screen 170 shown in FIG. 22, since the check box 172B is not displayed, it is also possible to make it impossible to select the “reduced picture A” physically.

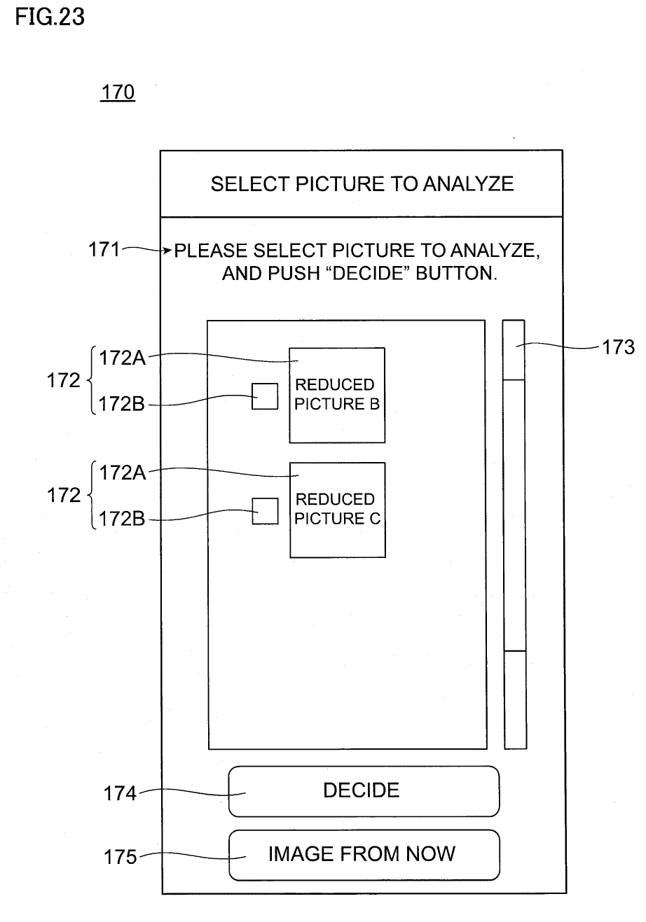

FIG. 23 illustrates another display example of the selection screen 170 used for selecting a picture of the analysis object. In FIG. 23, components corresponding to those of FIG. 18 are provided with same reference signs.

In case of FIG. 23, two reduced pictures 172A are displayed in the selection screen 170. Specifically, the “reduced picture B” and the “reduced picture C” are displayed in an order from top to bottom in FIG. 23. Note that, also in case of FIG. 23, a not-shown “reduced picture A” is included in the picture imaged within predetermined time. However, the not-shown “reduced picture A” is the picture which is selected as an object for reading the measured value in a previous operation.

Therefore, in the selection screen 170 shown in FIG. 23, the “reduced picture A” which is not a selection candidate, is excluded from an object for displaying. In other words, in the selection screen 170 shown in FIG. 23, only a candidate of selectable pictures is displayed.

When the selection screen 170 shown in FIG. 23 is used, it becomes impossible for the user to select physically the picture which is selected as an object for reading the measured value in a previous operation.

Note that, in the selection screen 170 shown in FIG. 23, a “Image from now” button 175 is displayed in addition to the “Decide” button 174 (refer to FIG. 18) used for deciding an analysis object. By adopting the “Image from now” button 175, also in the selection screen 170 of the picture to analyze, it is possible to select an image of a liquid crystal screen of a sphygmomanometer. This “Image from now” button 175 is also able to be adopted in the selection screen 170 shown in FIG. 18, FIG. 21 and FIG. 22.

(15) In the above-described exemplary embodiments, a case is assumed, which any kind of numerical value is read and displayed from a picture imaging the liquid crystal screen of the sphygmomanometer, even if the numerical value is wrong. However, in reality, there is also a case that the blood pressure value is not able to be read.

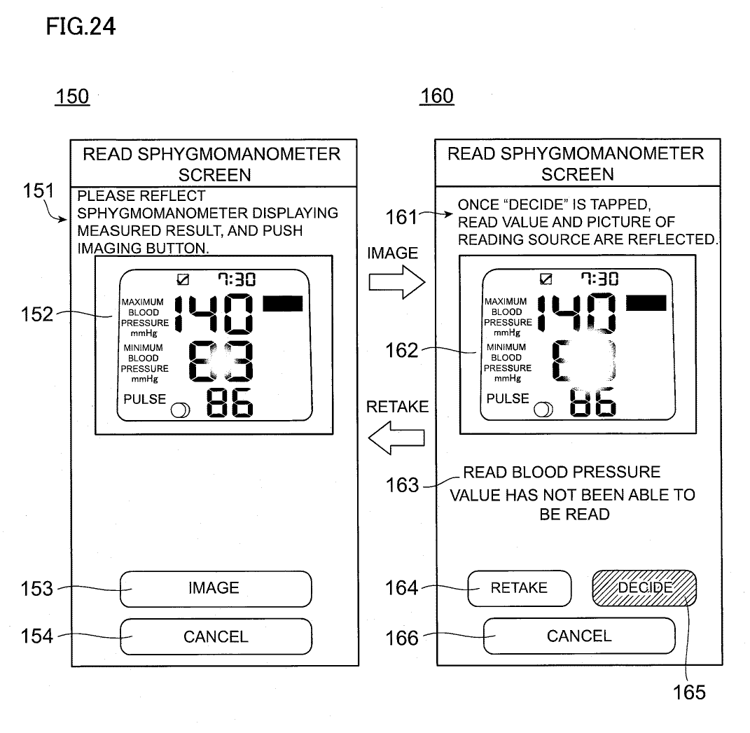

FIG. 24 illustrates another display example of the camera output screen 150 and the read value confirming screen 160. In FIG. 24, components corresponding to those of FIG. 14 are provided with same reference signs.

On the read value confirming screen 160 in FIG. 14, the blood pressure value and the pulse value read by an analysis of a picture are displayed in a field of the read value 163.

However, on the read value confirming screen 160 shown in FIG. 24, in the field of the read value 163, “Value has not been able to be read” is displayed. The display shown in FIG. 24 is displayed when even one of the numerical values which are display objects is not able to be read. Here, “Value has not been able to be read” is assumed to be a state that reliability of the numerical values read as a result of analyzing a picture, is lower than threshold. The reliability is calculated in the process of analyzing a picture, and output as a part of the result of analyzing the picture.

Note that, when even one of the numerical values which are display objects is not able to be read, the read numerical value may be displayed in a place where the numerical value is read, and a place where the numerical values is not able to be read may be blank, and separately, such as “There is a value that is not able to be read” may be displayed.

This displaying mode is an example of display that associates the effect that acquiring the measured value is failed with a picture.

This display makes it possible to notify a user of need for retaking a picture which is used for analyzing the measured value.

(16) In the above-described exemplary embodiments, a case is described, which is the case that when the imaging button 153 is operated in the camera output screen 150 (refer to FIG. 14), the read value confirming screen 160 (refer to FIG. 14) is displayed.

However, it needs to take several seconds (for example, 2 to 3 seconds) from the start of analyzing the picture until the analysis result (for example, blood pressure value) is displayed. Note that, this time is an example, and depends on, for example, processing capability of the user terminal 10 (refer to FIG. 1) or the like, quality of the picture which is an analysis object, and photographing environment. During analyzing the picture, the user only waits for the display of the analysis result. This waiting time (i.e. spare time) is waste time for the user.

Therefore, it is assumed that this spare time is used for describing how to use and operation procedure of the therapeutic app.

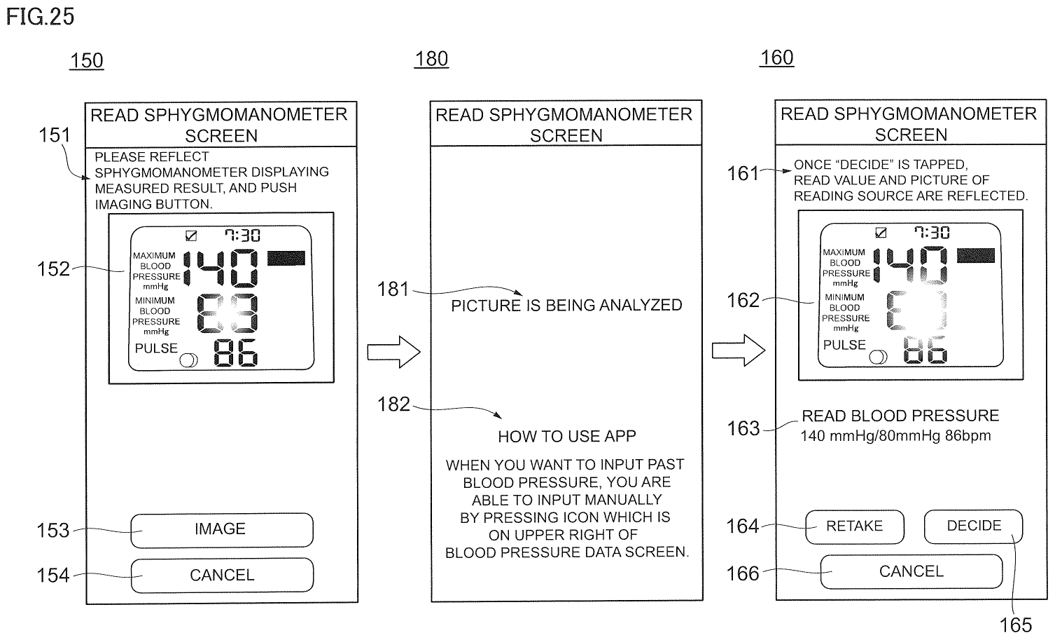

FIG. 25 illustrates a transition screen 180 displayed in a process of transitioning from the camera output screen 150 to the read value confirming screen 160. In FIG. 25, components corresponding to those of FIG. 14 are provided with same reference signs.

On the transition screen 180 shown in FIG. 25, a title of “Read sphygmomanometer screen” is attached.

In addition, in the transition screen 180 shown in FIG. 25, a displaying field 181 of processing state, and a message field 182 are placed. In the message field 182, for example, how to use the therapeutic app, points to be noted when measuring, timing of measuring, knowledge regarding a disease are displayed.

In case of FIG. 25, in the displaying field 181 of processing state, “Picture is being analyzed” is displayed. This display makes it possible for the user to understand the present processing state. As a result, even in case that the time until the blood pressure value is displayed is long, it is possible for the user to wait the display of the blood pressure value which is the analysis result without getting flustered.

In case of FIG. 25, in the message field 182, a title of “How to use app” is displayed, however, “Points to be noted when measuring”, “Timing of measuring”, “Knowledge regarding a disease” or the like are displayed corresponding to a displayed content.

In case of FIG. 25, as the “How to use app”, “When you want to input past blood pressure, you are able to input manually by pressing icon which is on upper right of blood pressure data screen” is displayed. The user who sees this display is able to know that it is possible to input the past blood pressure manually. Note that, in case of the above-described exemplary embodiments, it is possible to record the past blood pressure value or the like from a picture read from the picture folder.

Note that, it is desirable that the content of the information displayed in the message field 182 is different information every time for displaying. For example, based on the user's operation history, information which the frequency of the user's operation is low, may be a content for displaying preferentially. Furthermore, the displaying content may be changed randomly.

This displaying makes it possible to use effectively the spare time which is until the analysis result of the picture is displayed.

### Conclusion

The disclosure examples described in the above-described exemplary embodiments are shown below.

(((1)))

A non-transitory computer readable medium storing a program that causes a computer of a user terminal to execute; acquiring an analysis result of a picture which images a measurement instrument in every measurement session as a measured value, the measurement instrument measuring biological information, displaying the acquired measured value on a display section of the user terminal, associating with the picture of an acquiring source, and accepting an amendment of the measured value through an operation for the user terminal.

According to the program, it is possible to support a record of measured biological information. In addition, it is able to make it possible to amend the acquired measured value.

(((2)))

The non-transitory computer readable medium described in (((1))), wherein, in the accepting the amendment, an input of an amendment for the measured value is accepted. According to the program, it is possible to amend a numerical value displayed as an analysis result to correct value easily.

(((3)))

The non-transitory computer readable medium described in (((1))), wherein, in the accepting the amendment, a display of the display section is renewed based on a picture retaken according to an instruction of retaking operation, and a measured value acquired from the picture after retaking.

According to the program, even when a picture of the measured value is retaken, it is possible to make a set of the picture and the measured value, which are recorded corresponding to the measurement session, be a single set.

(((4)))

The non-transitory computer readable medium described in (((2))) or (((3))), wherein, in the accepting the amendment, a numerical value displayed on the display section at a time when an operation of finalizing a record of the measured value is accepted is recorded corresponding to the measurement session.

According to the program, it is possible to make a set of a picture and the measured value which are recorded corresponding to the measurement session, be a single set.

(((5)))

The non-transitory computer readable medium described in any one of (((1))) to (((4))), wherein, in the acquiring the analysis result, among stored pictures in a picture folder of the user terminal, a picture including the measurement instrument as a subject is extracted as an analysis object.

According to the program, it is possible to limit the number of pictures which are analysis objects.

(((6)))

The non-transitory computer readable medium described in any one of (((1))) to (((5)), wherein, in the acquiring the analysis result, plural pictures whose measurement sessions are different are extracted as the analysis object. According to the program, it is possible to reduce the number of recording works of the measured value.

(((7)))

The non-transitory computer readable medium described in any one of (((1))) to (((4))), wherein, in the acquiring the analysis result, among pictures imaged in the user terminal, a picture selected by a user is set as an analysis object.

According to the program, it is possible to limit the number of pictures which are analysis objects.

(((8)))

The non-transitory computer readable medium described in any one of (((1 to 6) wherein, in the acquiring the analysis result, a picture imaged within a period which goes back a predetermined amount of time from an activating time of the program is extracted as a processing object.

According to the program, it is possible to limit the number of pictures which are analysis objects.

(((9)))

The non-transitory computer readable medium described in any one of (((1))) to (6)) wherein, in the acquiring the analysis result, among pictures whose elapsed time from imaging time to present time is within a predetermined time, a picture which is not an object for acquiring until the present, is extracted as a processing object.

According to the program, it is possible to limit the number of pictures which are analysis objects.

(((10)))

The non-transitory computer readable medium described in any one of (((1))) to (((9))), wherein, in the acquiring the analysis result, the picture is provided to a server, and an analysis result by the server is acquired as the measured value.

According to the program, it is possible to improve accuracy of analysis of the measured value.

(((11)))

The non-transitory computer readable medium described in (((10))), wherein, in the acquiring the analysis result, the server is notified of a measurement item corresponding to the measured value which is a record object.

According to the program, it is possible to improve accuracy of analysis of the measured value.

(((12)))

The non-transitory computer readable medium described in any one of (((1))) to (((11))), wherein, in the acquiring the analysis result, when an output of measurement result by the measurement instrument is an analog form, the measured value is acquired through an analysis of the picture including a scale.

According to the program, it is possible to include an analog output measurement instrument in a processing object.

(((13)))

The non-transitory computer readable medium described in any one of (((1))) to (((12))), wherein, the program requires an input of a prescription code that is issued by a medical institution at a start of use.

According to the program, it is possible to make a habit of routine recording the measured value by a patient.

(((14)))

The non-transitory computer readable medium described in any one of (((1))) to (((13))), wherein, when acquiring the measured value, the program further includes displaying a candidate of a picture which is set as an analysis object, on the display section, and when displaying the candidate of the picture on the display section, among the candidate, a first candidate which has been used for acquiring the measured value is displayed with the effect that the first candidate has been used, or the first candidate is displayed in unselectable mode, or the candidate except for the first candidate is displayed on the display section.

According to the program, it is possible to prevent reacquiring the measured value from the picture which has been used for analysis of the measured value.

(((15)))

The non-transitory computer readable medium described in any one of (((1))) to (((14))), wherein the program further includes displaying an average value of plural measured values acquired from plural pictures corresponding to time zone, as the measured value in the time zone corresponding to the time when the plural pictures imaged within a predetermined time are imaged.

According to the program, it is possible to display the average value of the measured values corresponding to each time zone automatically. For example, even in case the measured value of one day morning is recorded in night of the same day, or another day, it is possible to display the average value of the measured values associating with the time zone corresponding to the time when each picture is imaged.

(((16)))

The non-transitory computer readable medium described in any one of (((1))) to (((14))), wherein the program further includes displaying the effect that the measured value has failed to be acquired, associating with the picture, when the measured value is not able to be acquired from the picture by the analysis.

According to the program, it is possible to notify the user of need for retaking a picture which is used for analyzing the measured value.

(((17)))

The non-transitory computer readable medium described in any one of (((1))) to (((14))), wherein the program further includes displaying information regarding at least one in how to use the program, points to be noted when measuring, timing of measuring and knowledge regarding a disease in a period from the start of analyzing the picture until the analysis result is acquired.

According to the program, it is possible to use effectively the spare time which is until the analysis result of the picture is displayed.

The foregoing description of the exemplary embodiment of the present invention has been provided for the purposes of illustration and description. It is not intended to be exhaustive or to limit the invention to the precise forms disclosed. Obviously, many modifications and variations will be apparent to practitioners skilled in the art without departing from the scope and spirit of the present invention. The exemplary embodiment was chosen and described in order to best explain the principles of the invention and its practical applications, thereby enabling others skilled in the art to understand the invention for various embodiments and with the various modifications as are suited to the particular use contemplated. It is intended that the scope of the invention be defined by the following claims and their equivalents.

What is claimed is:

1. A non-transitory computer readable medium storing a program that causes a computer of a user terminal to execute;
acquiring an analysis result of a picture which images a measurement instrument in every measurement session as a measured value, the measurement instrument measuring biological information, displaying the acquired measured value on a display section of the user terminal, associating with the picture of an acquiring source, and accepting an amendment of the measured value through an operation for the user terminal.

2. The non-transitory computer readable medium according to claim 1, wherein, in the accepting the amendment, an input of an amendment for the measured value is accepted.

3. The non-transitory computer readable medium according to claim 1, wherein, in the accepting the amendment, a display of the display section is renewed based on a picture retaken according to an instruction of retaking operation and a measured value acquired from the picture after retaking.

4. The non-transitory computer readable medium according to claim 2, wherein, in the accepting the amendment, a numerical value displayed on the display section at a time when an operation of finalizing a record of the measured value is accepted is recorded corresponding to the measurement session.

5. The non-transitory computer readable medium according to claim 3, wherein, in the accepting the amendment, a numerical value displayed on the display section at a time when an operation of finalizing a record of the measured value is accepted is recorded corresponding to the measurement session.

6. The non-transitory computer readable medium according to claim 1, wherein, in the acquiring the analysis result, among stored pictures in a picture folder of the user terminal, a picture including the measurement instrument as a subject is extracted as an analysis object.

7. The non-transitory computer readable medium according to claim 6, wherein, in the acquiring the analysis result, a plurality of pictures whose measurement sessions are different is extracted as the analysis object.

8. The non-transitory computer readable medium according to claim 1, wherein, in the acquiring the analysis result, among pictures imaged in the user terminal, a picture selected by a user is set as an analysis object.

9. The non-transitory computer readable medium according to claim 1, wherein, in the acquiring the analysis result, a picture imaged within a period which goes back a predetermined amount of time from an activating time of the program is extracted as a processing object.

10. The non-transitory computer readable medium according to claim 1, wherein, in the acquiring the analysis result, among pictures whose elapsed time from imaging time to present time is within a predetermined time, a picture which is not an object for acquiring until the present is extracted as a processing object.

11. The non-transitory computer readable medium according to claim 1, wherein, in the acquiring the analysis result, the picture is provided to a server, and an analysis result by the server is acquired as the measured value.

12. The non-transitory computer readable medium according to claim 11, wherein, in the acquiring the analysis result, the server is notified of a measurement item corresponding to the measured value which is a record object.

13. The non-transitory computer readable medium according to claim 1, wherein, in the acquiring the analysis result, when an output of measurement result by the measurement instrument is an analog form, the measured value is acquired through an analysis of the picture including a scale.

14. The non-transitory computer readable medium according to claim 1, wherein the program requires an input of a prescription code that is issued by a medical institution at a start of use.

15. The non-transitory computer readable medium according to claim 1, wherein, when acquiring a measured value, the program further comprises displaying a candidate of a picture which is set as an analysis object, on the display section, and when displaying the candidate of the picture on the display section, among the candidate, a first candidate which has been used for acquiring a measured value is displayed with the effect that the first candidate has been used, or the first candidate is displayed in unselectable mode, or the candidate except for the first candidate is displayed on the display section.

16. The non-transitory computer readable medium according to claim 1, wherein the program further comprises displaying an average value of a plurality of measured values acquired from a plurality of pictures corresponding to time zone, as a measured value in the time zone corresponding to the time when a plurality of pictures imaged within a predetermined time is imaged.
